(19) United States
(12) Patent Application Publication
(10) Pub. No.: US 2026/0306738 A1
(43) Pub. Date: Oct. 8, 2026

(54) METHOD AND APPARATUSES FOR DEPLOYING A DEPLOYABLE ELEMENT OF A WEARABLE MEDICAL DEVICE

(71) Applicant: INSULET CORPORATION, Acton, MA (US)

(72) Inventors: John BUSSIERE, Littleton, MA (US); Lucas MCGAHREN, Somerville, MA (US); Ian MCLAUGHLIN, Groton, MA (US); John D'ARCO, Wilmington, MA (US)

(21) Appl. No.: 19/638,726

(22) Filed: Apr. 3, 2026

Related U.S. Application Data
(60) Provisional application No. 63/783,170, filed on Apr. 3, 2025.

(51) Int. Cl. A61M 5/142

(52) U.S. Cl. CPC A61M 5/14248; A61M 2005/14252; A61M 2202/0007; A61M 2202/04; A61M 2205/103; A61M 2205/3303; A61M 2205/3327; A61M 2205/50; A61M 2205/8212; A61M 2210/04

(57) ABSTRACT

Exemplary embodiments provide methods, apparatuses, and supporting structures for deploying a deployable element (such as a cannula or subcutaneous sensor) into a user's tissue (such as through skin tissue). Front-end electronics may be provided on a support structure and may move while the deployable element is deployed. A flexible connector can connect the front-end electronics to back-end electronics (e.g., a main processor circuit of an associated body-worn device).

### CROSS-REFERENCE TO RELATED APPLICATION

This application claims priority to U.S. Patent Application Ser. No. 63/783,170, filed on Apr. 3, 2025, the contents are incorporated herein in their entirety. This application is also related to U.S. patent application Ser. No. 18/390,450, filed on Dec. 20, 2023, which claims priority to U.S. Patent Application Ser. No. 63/434,243, filed on Dec. 21, 2022, the contents of each of which are incorporated herein in their entirety.

### BACKGROUND

Advances in miniaturization have allowed for the creation of body-worn devices capable of administering therapeutics, as well as sensing and reporting physiological characteristics. Examples of such devices include (but are not limited to) devices for dispensing insulin, glucagon-like peptide (GLP-1), a combination of insulin and GLP-1, fertility drugs, white blood cell stimulating drugs, or other medicaments, and sensor devices containing a sensor element for sensing glucose, ketones, or more generally, analytes, hormones, or other physiological factors.

Dispensing devices generally deploy a needle, cannula, conduit, or other element into the user's tissue (e.g., through the user's skin into the subcutaneous space). Sensor devices generally do not deploy a sensor element themselves, but use a separate inserter (which is often disposed of after a single insertion or use) for the sensor element and then connect to the sensor element after it is inserted into the user's tissue. The separate inserter is often made up of bulky plastic material and is often disposed of after a single insertion or use, leading to significant environmental waste. The sensor device is made up of electronic elements that connect to the sensor element after the sensor element has been inserted, and these electronic elements typically comprise a printed circuit board and communication elements such as an antenna and radio that allow the signals sensed by the sensor element to be converted into a usable format and transmitted to another device, such as a medicament dispensing device, that is typically a separate device than the sensor device and spaced apart from the sensor device on the user's body. The medicament dispensing device may be referred to as an automated medicament delivery device, or AMD device, such as an automated insulin delivery device, or AID device.

As discussed and shown in U.S. patent application Ser. No. 18/390,450, the dispensing device and the sensor device may be co-located in one device, though they may preferably have separate elements penetrating the user's skin: for example, a cannula for the dispensing device and a sensor element for the sensor device. The dispensing device and the sensor device, or the corresponding deployable elements of those devices, may be located on opposite sides of the single, wearable device (i.e., a co-located device). For example, the dispensing cannula may be located on one side of the wearable device and the sensor element may be located on another side of the wearable device.

Rather than have a standalone inserter (or inserters) that is disposed of immediately after use, the inserter (or inserters) can be incorporated into the single, wearable device. One inserter can be used for the sensor element and one inserter can be used for the dispensing element (e.g., a cannula). A similar inserter can be used in both instances. For the sake of brevity in this application, the element that is deployed will be generally referred to as a deployable element, though it should be understood that the term deployable element encompasses sensor elements, needles, cannulas, and other similar deployable elements.

As described in U.S. patent application Ser. No. 18/390,450, the deployable element needs to be sufficiently rigid to penetrate the skin without buckling, if it is deployed without a supportive device or structure. As these deployable elements become narrower or more flexible and/or slender, they trend towards becoming insufficiently stiff due to intrinsic material physical properties (e.g., the elastic modulus) and very small physical sizes and geometries (e.g., geometric section stiffness). Thin, flexible, slender elements that naturally have low geometric stiffness are notoriously difficult to deploy to appropriate penetration depths into deformable materials such as human skin tissues. They instead trend towards structural compromise due to axial compressive forces, which can result in undesired transverse displacements (e.g., buckling or deflection, such as by bouncing off the top surface of the skin and failing to penetrate, as when the deployable element is inserted at an angle) of the structure under critical load conditions or upon insertion or penetration through human skin, for example.

Moreover, when the sensor device and the dispensing device are co-located on one device, various elements that may be required in each separate device may serve both the sensor device (or sensor element) and dispensing device when such devices are co-located in a single device. For example, the number of printed circuit boards, microchips, memory, antennas, amplifiers, wires, batteries, and other elements may be reduced when the sensor device and dispensing device are co-located in one device; for example, in one wearable device on one location on the user's body.

### BRIEF SUMMARY

Exemplary embodiments provide unique deployment assemblies configured to mitigate deployment failures during penetration events and allow for efficient and proper functions of use after deployment.

Some examples provide front-end electronics that connect to a deployable element that is inserted into the user's tissue while front-end electronics themselves remain outside of the skin of the user. The front-end electronics may include amplifiers, converters, as well as other types of electronics to perform initial processing on the signal received from the deployable element. The processed signal may then be passed to back-end electronics that are located off of the moving support element attached to the deployable element.

Further examples, which may be employed together or separately with the front-end electronics configuration noted above, provide a flexible connector that connects the front-end electronics to back-end electronics. The flexible connector may include a flexible end supported by heat stakes or similar structures providing strain relief as the support structure holding the front-end electronics rotates or translates. The other end of the flexible connector may be a rigid end that can secure that end to the back-end electronics with a simple heat stake (e.g., without the need for soldering or welding). Alternatively, the other end may be flexible, in which case the end of the flexible connector may be welded or soldered to contacts on the back-end electronics board.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A is a perspective view an exemplary rotary deployment mechanism according to a first embodiment.

FIG. 1B is a side view an exemplary rotary deployment mechanism according to a first embodiment.

FIG. 1C is a perspective view of an exemplary rotary deployment mechanism according to a second embodiment.

FIG. 1D depicts an exemplary dampener suitable for use with exemplary embodiments.

FIG. 1E depicts an internal view of an exemplary dampener suitable for use with exemplary embodiments.

FIG. 1F is a side view of an exemplary rotary deployment mechanism with a dampener.

FIG. 2 is a perspective view depicting the sheave assembly in a first, undeployed state.

FIG. 3 is a perspective view depicting the sheave assembly in a second, deployed state.

FIG. 4A-FIG. 4D provide various closeup views of the sheave assembly of the first embodiment.

FIG. 5 provides various views of the deployment mechanism of the first embodiment enclosed in a standalone housing.

FIG. 6 provides various views of the deployment mechanism of the first embodiment enclosed in a housing capable of accommodating other components, such as an automated insulin delivery (AID) device.

FIG. 7A is a perspective exploded view of an exemplary rotary deployment mechanism according to a first embodiment.

FIG. 7B depicts a perspective view from the opposite side as compared to FIG. 7A.

FIG. 8 is a side view depicting the deployable element 102 of the first embodiment in more detail.

FIG. 9 is a side view depicting an alternate configuration for the deployable element 102 of the first embodiment.

FIG. 10A is a front and side perspective view depicting the sheave assembly of the first embodiment in more detail.

FIG. 10B is a side and rear perspective view depicting the sheave assembly of the first embodiment in more detail.

FIG. 11 is a perspective view showing electrical connections between the sensor and onboard front-end electronics, and an electrical service loop for connecting the front-end electronics to back-end electronics in a device main body.

FIG. 12 depicts an exemplary architecture for a deployable element support including front-end electronics.

FIG. 13-FIG. 15 depict an exemplary flexible connector for connecting front-end electronics attached to a deployable element with a back-end circuit in a device main body.

FIG. 16-FIG. 17 depict an exemplary version of the flexible connector connecting a deploying or movable element to a non-deploying or fixed element.

FIG. 18 depicts an exemplary heat stake for connecting the flexible connector to back-end electronics.

FIG. 19 illustrates an exemplary heat stake for connecting the flexible connector to front-end electronics.

FIG. 20A-FIG. 21 depict an exemplary configuration of a heat stake connection configured to constrain the connector end to improve an electrical connection between the connector and the back-end electronics.

FIGS. 22A-22D depict an alternative release mechanism suitable for use with exemplary embodiments.

FIG. 23 is a perspective exploded view of an exemplary linear deployment mechanism according to a second embodiment.

FIG. 24 is a side- and bottom-perspective view of the assembled linear deployment mechanism of the second embodiment in a first, undeployed state.

FIG. 25 is a side- and bottom-perspective view of the assembled linear deployment mechanism of the second embodiment in a second, deployed state.

FIG. 26 depicts the introducer of the second embodiment in more detail.

FIG. 27 is a side view of a structure for tensioning a user's epidermis suitable for use with any of the described embodiments.

FIG. 28 is a side view of an alternative epidermal tensioning structure suitable for use with any of the described embodiments.

FIG. 29 is a perspective view of an epidermal tensioning structure.

FIG. 30 is a side view of the epidermal tensioning structure of FIG. 27 showing different possible protrusion profile shapes.

FIG. 31A is a perspective view depicting an exemplary structure suitable for use with prior described embodiments.

FIG. 31B illustrates an aspect of the subject matter in accordance with one embodiment.

FIG. 31C illustrates an aspect of the subject matter in accordance with one embodiment.

FIG. 32 is a side view of the exemplary structure of FIG. 31A.

FIG. 33 is a perspective view depicting an exemplary structure suitable for use with prior described embodiments.

### DETAILED DESCRIPTION

Exemplary embodiments provide methods, apparatuses, and supporting structures for deploying a deployable element (such as a cannula or a sensor) into a user's tissue (such as skin tissue).

A first embodiment leverages rotary motion to deploy the element while constraining at least part of the element for at least part of the motion to prevent buckling. The constraints may allow the element to be deployed without the assistance of an introducer that supports the deployable element.

Utilizing rotary motion derived from a source of harnessed potential energy (e.g., a torsion spring) and leveraging the moment of a force applied to a selectively constrained element at the radius of a flywheel or sheave, the thin element is guided with minimum frictional resistance in a circumferential-to-linear motion and forced into the skin tissue layers at the distal end of the deployable element, which may be a sharpened end. Such circumferential wrapping and initial containment provide for a neatly packaged (minimal spatial volume of mechanism) deployment method of deployable elements (such as a sensor element) to depths of subcutaneous tissue below the outer surface of skin (in some embodiments, about a 4-7 mm depth).

A second embodiment leverages linear motion to deploy the flexible, slender element with the assistance of an introducer element to provide support. Utilizing linear motion derived from a source of harnessed potential energy (e.g., a helical compression spring) and leveraging of a force applied to selectively constrained configuration of a single or multiple thin elements, the thin element(s) is guided with minimum frictional resistance in a linear motion and forced into the skin tissue layers initiated at the distal sharpened end of the slender element (a subcutaneous introducer in this embodiment). Such linear containment provides for a neatly packaged (minimal spatial volume of mechanism) deployment method of deployable elements (such as a sensor element) to depths of subcutaneous tissue below the outer surface of skin (4-7 mm).

Other embodiments provide support features that improve the functionality of the above-mentioned embodiments. For instance, a sacrificial sleeve may impermanently hold or embrace the deployable element to temporarily increase the structural stiffness of the deployable element.

Another support structure may provide for tensioning the user's tissue, such as by causing the skin to bulge in an area where deployment of a deployable element is facilitated. This structure may include a protruding annulus structure on a body worn or applied device in contact, and/or adhered, with the skin during insertion of the deployable element into the human skin layers.

The deployable element (e.g., cannula or sensor) itself may also be structured or configured to support deployment. For instance, laminated composition of polymers, metals, and other materials may be combined into varied cross-sectional shapes. These elements may be employed to develop desired directional stiffness for improved deployment penetration and/or to maintain electrically conductive signal conduit/traces to embedded electronics hardware.

### EXEMPLARY EMBODIMENTS

As an aid to understanding, a series of examples will first be presented before detailed descriptions of the underlying implementations are described. It is noted that these examples are intended to be illustrative only and that the present invention is not limited to the embodiments shown.

Reference is now made to the drawings, wherein like reference numerals are used to refer to like elements throughout. In the following description, for purposes of explanation, numerous specific details are set forth in order to provide a thorough understanding thereof. However, the novel embodiments can be practiced without these specific details. In other instances, well known structures and devices are shown in block diagram form in order to facilitate a description thereof.

In the Figures and the accompanying description, the designations “a” and “b” and “c” (and similar designators) are intended to be variables representing any positive integer. Thus, for example, if an implementation sets a value for a=5, then a complete set of components 122 illustrated as components 122-1 through 122-a may include components 122-1, 122-2, 122-3, 122-4, and 122-5. The embodiments are not limited in this context.

### Introducer-Less Rotary Deployment Mechanism

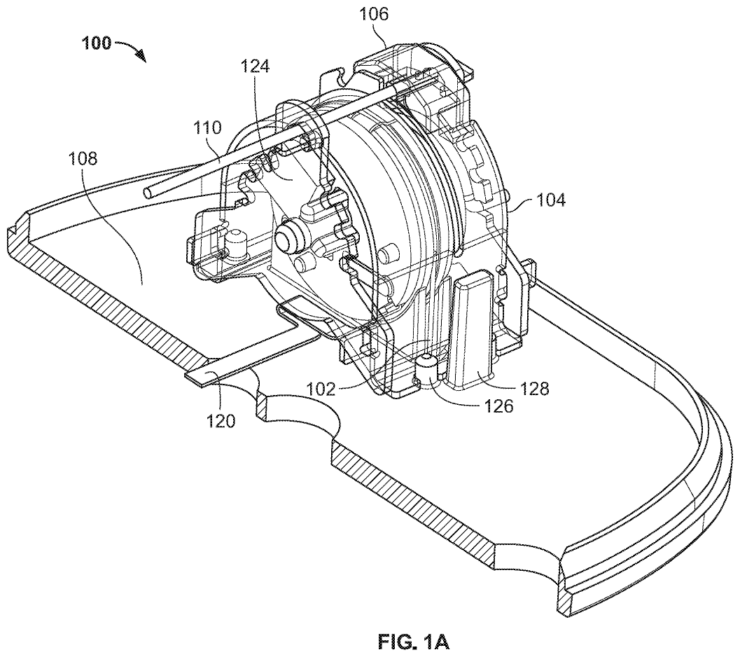

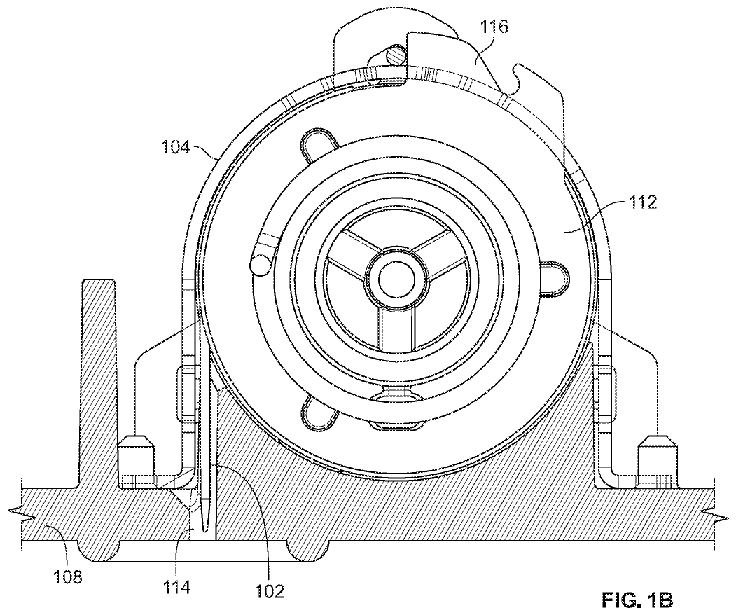

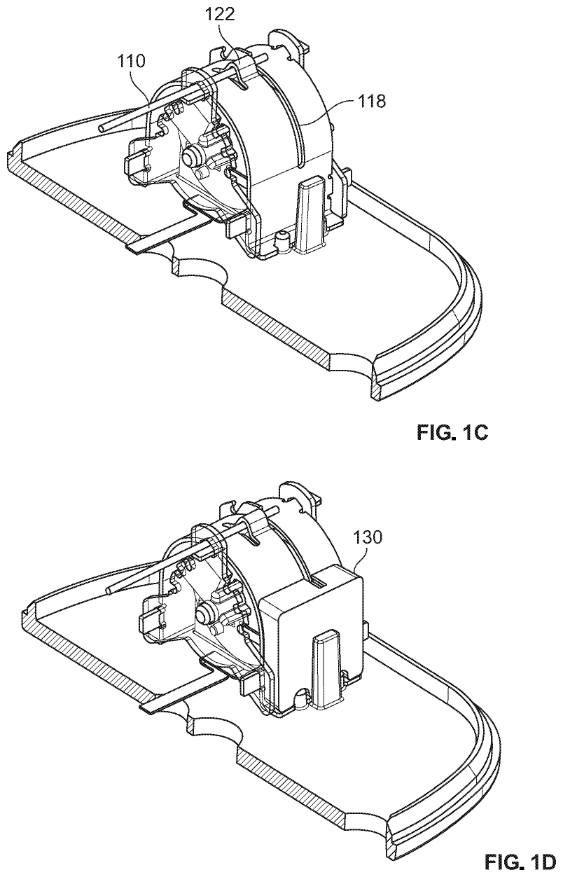

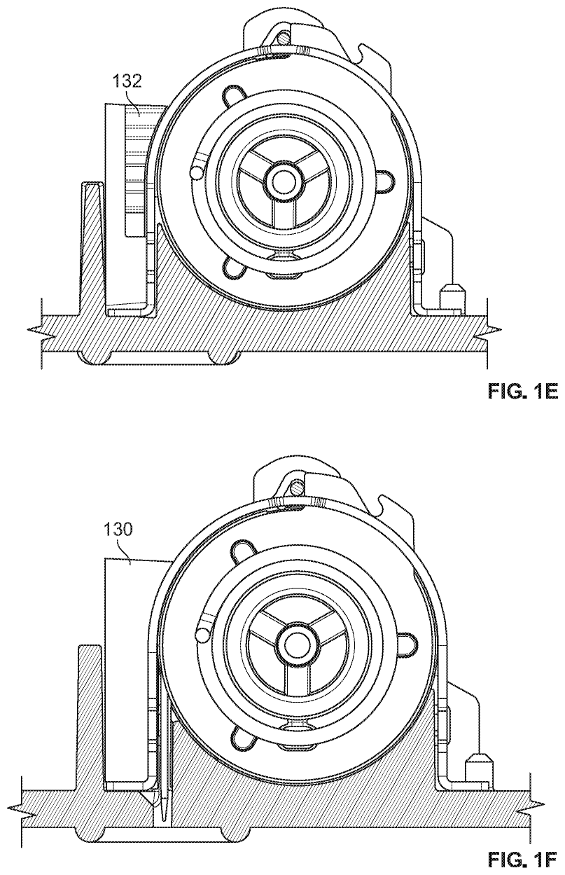

A first embodiment provides an introducer-less rotary deployment assembly 100, as shown in FIG. 1A-FIG. 4D. The deployment assembly 100 may be used to deploy a deployable element, such as a cannula, a sensor, or both (as appropriate). A “sensor” is a component configured to detect or measure a physical, chemical, or biological parameter and to generate a corresponding signal representative of the detected parameter, the sensor comprising a sensing element that directly interacts with the parameter to be detected. The sensor may include electrical or electronic components configured to generate, condition, or otherwise derive the signal from the sensing element. However, the term “sensor” is used in distinction from general-purpose processing or control electronics that are arranged to further process or interpret the signal beyond its generation from the detected parameter.

FIG. 1A and FIG. 1B depict the assembled deployment assembly 100. The deployment assembly 100 comprises a deployable element 102, which may be a cannula, needle, conduit, sensor, etc. For instance, the deployable element 102 may be a cannula or needle for delivering a medicament such as insulin. The deployable element 102 may be a glucose sensor or ketone sensor (e.g., an electrochemical sensor; a fiber optic sensor, a wire sensor, an optofluorescence sensor, etc.), a temperature sensor, or an activity sensor. In some embodiments, multiple sensors may be combined into a single deployable element 102, as might be the case where one side of the deployable element 102 serves as a glucose sensor and the opposite side serves as a ketone sensor.

In this example, the deployable element 102 rotates along the outer radius of a flywheel or sheave until the rotational motion is converted to linear motion and the deployable element 102 moves linearly into or out of an opening 114 in a base 108 that supports the deployment assembly 100 (e.g., on the skin of the user). The deployable element 102 may be selectively constrained by the cowling 104 that surrounds the deployable element 102. Additionally or alternatively, the cowling 104 may take the form of a housing for the conduit deployment assembly 100.

The deployment assembly 100 may be positioned and/or retained on the base 108 using one or more base protrusions 126 and/or ribs 128 that fit into corresponding cutouts in the bottom of the cowling 104, which may be flared outwards to allow for a connection to be made between the deployment assembly 100 and the base 108.

Utilizing rotary motion derived from a source of harnessed potential energy (in this embodiment a torsion spring controlled by a release 110 fitting into a bearing block 106) and leveraging the moment of a force applied to the deployable element 102, the deployable element 102 is guided with minimum frictional resistance in a circumferential to linear motion and forced into the skin tissue layers at the distal end of the deployable element 102 (a subcutaneous sensor in this application, but alternatives may be used as explained above and below). Such circumferential wrapping and initial containment allow for a mechanism with a relatively small spatial volume (e.g., less than about 0.25″×0.25″×0.375″ (6.35 mm×6.35 mm×9.525 mm)).

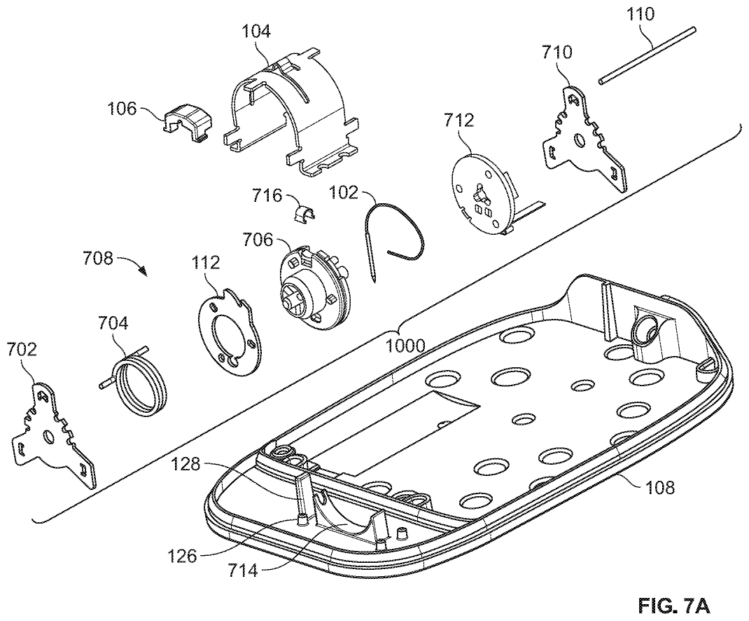

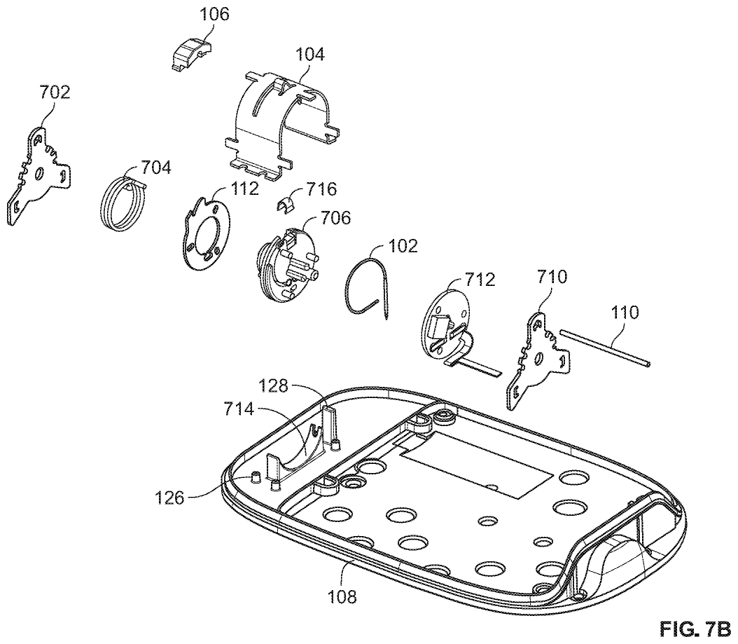

The deployment assembly 100 allows a sensor or other elements to be deployed to depths of subcutaneous tissue below the outer surface of skin (e.g., ~4-7 mm). The depth of penetration may be controlled by the shape of a stop plate 112 mounted to the sheave. As shown in FIG. 1B, FIG. 1C, FIG. 7A and FIG. 7B, the stop plate 112 includes a protrusion 116 that fits into a groove 118 in the cowling 104, and further includes openings allowing the stop plate 112 to be mounted to the sheave. When the sheave turns, the stop plate 112 rotates and the protrusion 116 moves within the groove 118 until hitting a stopping point at the end of the groove 118, which prevents the sheave from rotating further (and therefore stops the linear motion of the deployable element 102).

In the case where the deployable element 102 is a sensor, the sensor may relay signals back to integrated front-end electronics 124 on board the deployment assembly 100. The integrated front-end electronics 124 may include a processor, amplifiers (fixed or programmable), filtering, multiplexing, converters (such as an analog-to-digital and digital to analog converters), ESD suppressors, and/or memory. “Front-end electronics” refer to electronic components that are co-located with the deployable element or located at a position closer to the deployable element than back-end electronics; thus, the front-end electronics 124 may be arranged further in the deployment direction than the back-end electronics after deployment. In many embodiments, the front-end electronics provide at least signal amplification and/or signal-to-noise ratio improvement capabilities. Some limited front-end-processing may be performed on the sensor signals by the integrated front-end electronics 124. The resulting data may be transmitted via a service portion 120 to the processor on the body-worn device.

In the embodiments of FIG. 1A and FIG. 1B, the release 110 is secured in a bearing block 106 until it is pulled away from the stop plate 112. FIG. 1C depicts an alternative configuration where the release 110 is secured in a cowling support 122 formed in the cowling 104. The cowling support 122 may simply be a raised loop formed from the material of the cowling 104, or may be a separate element attached to the cowling 104.

FIG. 1D-FIG. 1F depict an optional damper 130 suitable for use with exemplary embodiments. For example, the damper 130 may be a viscoelastic damper attached to the cowling 104 and filled with a viscous medium to absorb energy from the deployment assembly 100. The damper 130 may include one or more ridges 132 or feathered elements that the deployable element 102 may wrap through, where the size and shape of the ridges 132 configured to slow down the motion of the spring that deploys the deployable element 102. In some embodiments, the ridges 132 may be set in a configuration to act only on a part of the stroke that deploys the deployable element 102 (such as the last ½-¼ of the stroke).

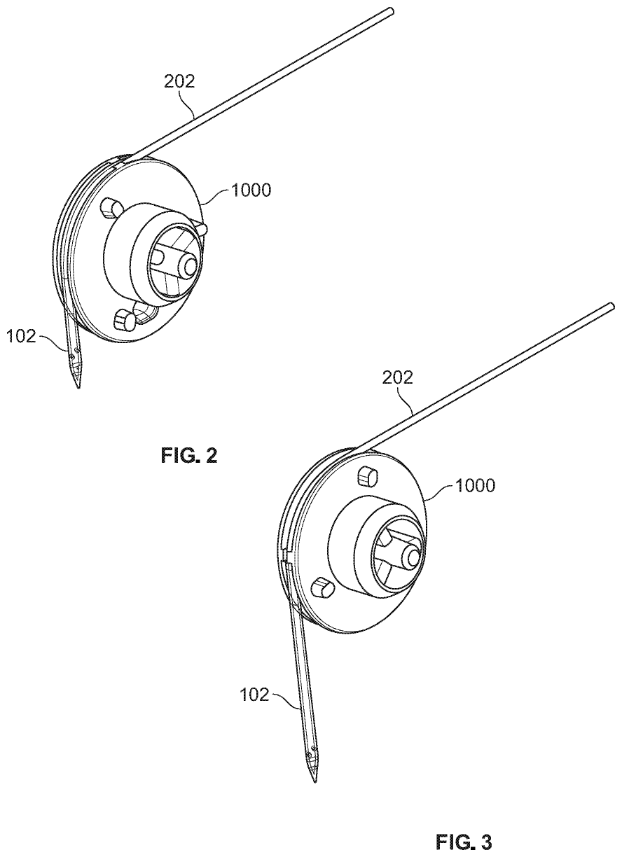

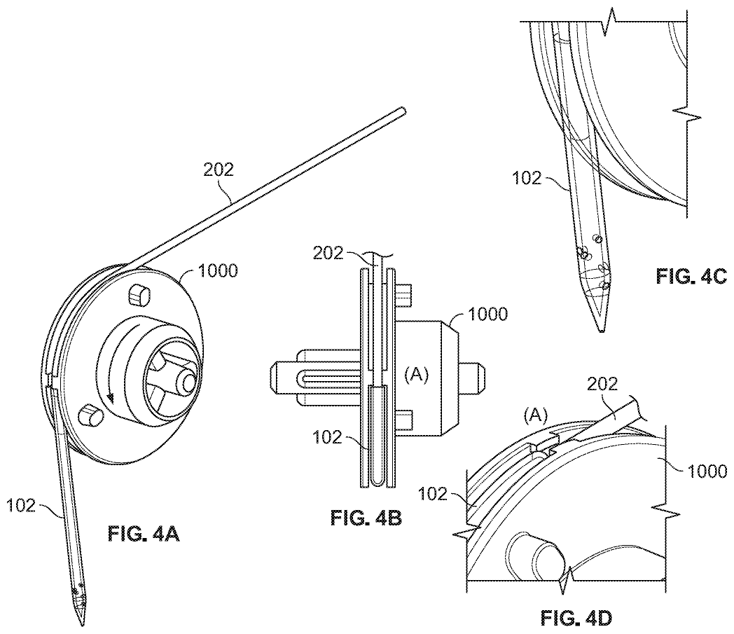

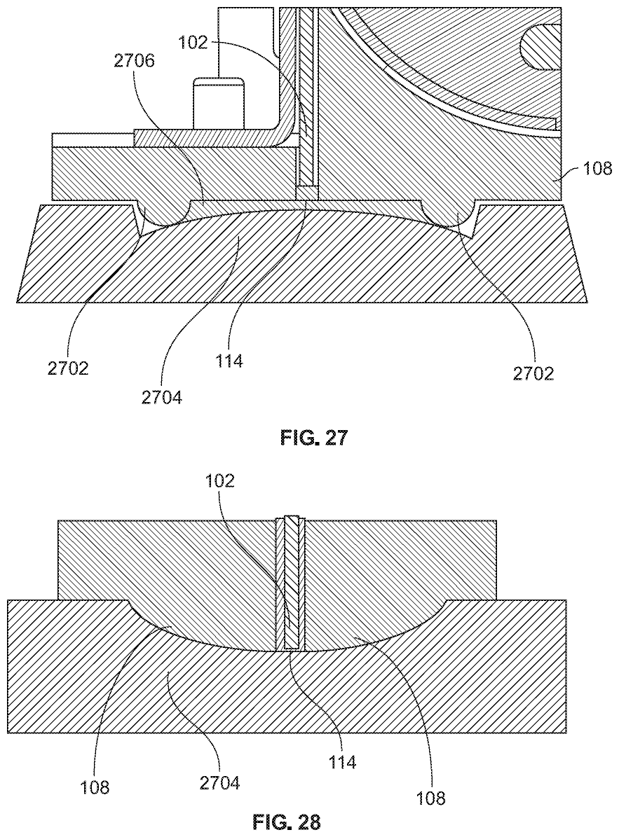

FIG. 2, FIG. 3, and FIG. 4A-FIG. 4D show the deployment of an exemplary deployable element 102. In an initial state (FIG. 2 and FIG. 4C), the deployable element 102 is undeployed and withdrawn through the opening 114 (shown, for example, in FIG. 27). As the sheave assembly 1000 rotates, it drives the deployable element 102 in a linear motion (downward, in FIG. 2, FIG. 3, and FIG. 4A) until the deployable element 102 reaches its fully deployed extent (FIG. 3 and FIG. 4A).

FIG. 2, FIG. 3, FIG. 4A, FIG. 4B, and FIG. 4D further show an optional fluid conduit 202, which may be used when the deployable element 102 represents a cannula, conduit, or needle for delivering a drug (such as liquid insulin). The fluid conduit 202 connects to the deployable element 102 (e.g., through a flexible, elastic, tapered, or extensible portion that allows the interface between the deployable element 102 and fluid conduit 202 to move as the sheave rotates, or by moving the length of the fluid conduit 202 into/out of an attached reservoir). The fluid conduit 202 may connect to a reservoir and allow fluid to be delivered from the reservoir through the deployable element 102. The fluid conduit 202 may form a bend when the deployable element 102 is in its undeployed or initial state (FIG. 2 and FIG. 4C) and this bend may have the necessary slack to allow the deployable element 102 to deploy without interrupting fluid connections from the reservoir to the fluid conduit 202 to the deployable element 102. The deployable element 102 may have a sharpened distal end, an open lumen at its distal end fluidly connected to fluid conduit 202, and/or one or more holes proximate to its distal end (e.g., as shown in FIG. 4C) for delivering a medicament such as insulin.

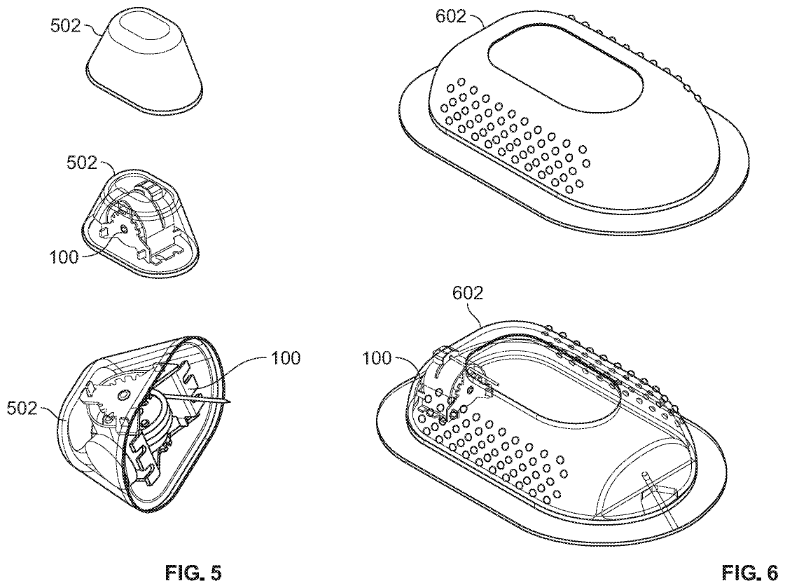

Due to the use of rotary motion to drive the deployable element 102, the deployment assembly 100 can be held to such a small size. Accordingly, it can be deployed in a relatively small housing, either on its own (FIG. 5) or together with other elements of a drug-delivery device (e.g., an automated insulin delivery, or “AID,” device; see FIG. 6).

In the standalone example of FIG. 5, the deployment assembly 100 fits within its own assembly housing 502. In this form factor, the deployment assembly 100 could be used, for example, to deploy a standalone sensor (such as a glucose sensor for a continuous glucose monitor).

In the combined example of FIG. 6, the deployment assembly 100 is contained within a larger unitary device housing 602 that may also house other components of a drug-delivery device. For example, the deployment assembly 100 may be used to deploy a glucose sensor or ketone sensor (as in FIG. 5) that is used to control the delivery of metered amounts of insulin from an AID device. A dispensing device, such as an AID device, may also be contained within the unitary device housing 602. Separately or together with the glucose monitor, the AID device may also make use of a deployment assembly 100 to deploy a needle, cannula, or conduit for delivering the insulin or other medicament.

FIG. 7A-FIG. 7B depict exploded views of the various components of the deployment assembly 100 from different perspectives.

As previously noted, the deployment assembly 100 sits on a base 108 that is shaped to hold a sheave assembly 1000. In particular, the base 108 includes a semicircular rest configured to support a sheave assembly 1000.

The sheave assembly 1000 may be controlled by a release 110, which includes a curved portion that fits within a bearing block 106. The bearing block 106 connects to a first bearing plate 702 that is fixed to a first end of the cowling 104, thereby holding the bearing block 106 in place. The release 110 restrains the protrusion 116 of the stop plate 112. When the release 110 is pulled (see FIG. 1A), the curved portion is pulled out of the bearing block 106 into the vicinity of the protrusion 116, thereby releasing the protrusion 116 (and, by extension the stop plate 112) from being restrained by the release 110.

The release 110 may be manually activated by the user. Alternatively, the release 110 may be activated by a shape memory alloy (SMA) wire that changes length in response to an outside stimulus (e.g., an application of an electric current or a change in temperature). The SMA wire may be activated by circuitry that, for example, responds to a transmitter/receiver in the deployment assembly 100 indicating that it is time to deploy the deployable element 102 (as might be the case if the deployment assembly 100 is part of a drug delivery device controlled, e.g., by a signal from a mobile phone or similar device), by a button press, etc. Alternatively, the release 110 may be manually triggered by a user, such as by pushing a button at an outside surface of the unitary device housing 602 (such as a housing of an insulin pump) or pulling a prong or the release 110 itself, which may be accessible to a user at an outside surface of unitary device housing 602. In this latter example, release 110 may be removed entirely by a user while triggering insertion of deployable element 102, and may be discarded by the user. In an alternative embodiment, release 110 may be connected to a gearing mechanism and/or a plunger element in a reservoir of a drug delivery device that is driven by a gearing mechanism. As the gearing mechanism advances, it may reach a threshold point where the release 110 is pulled or pushed so as to cause deployment of the deployable element 102.

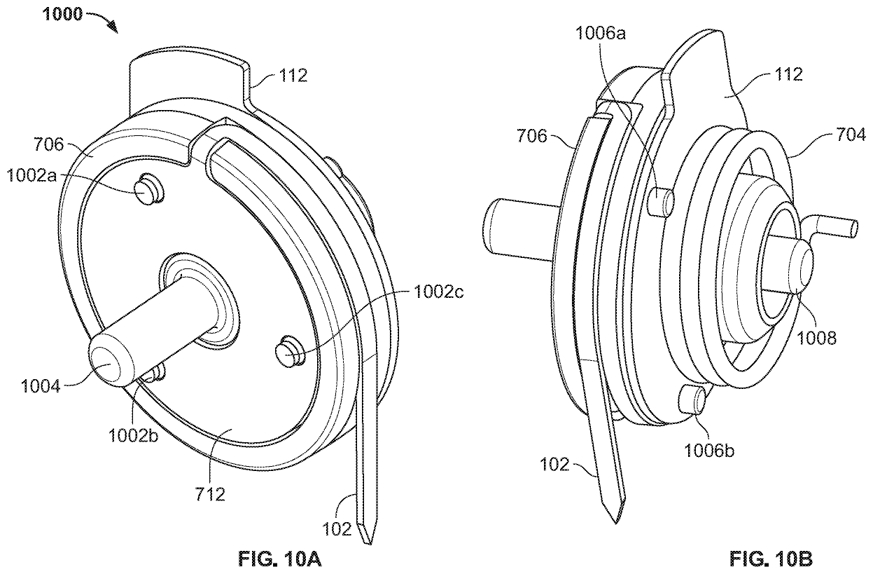

The stop plate 112 is fixed to a sheave 706 (see FIG. 10A and FIG. 10B for details) that receives energy from a spring 704 (in this example, a torsion or coil spring). When the stop plate 112 is released, the sheave 706 turns under the action of the spring 704, causing the protrusion 116 to rotate inside a groove in the cowling 104. When the protrusion 116 reaches the end of the groove, the stop plate 112 is restrained and ceases moving.

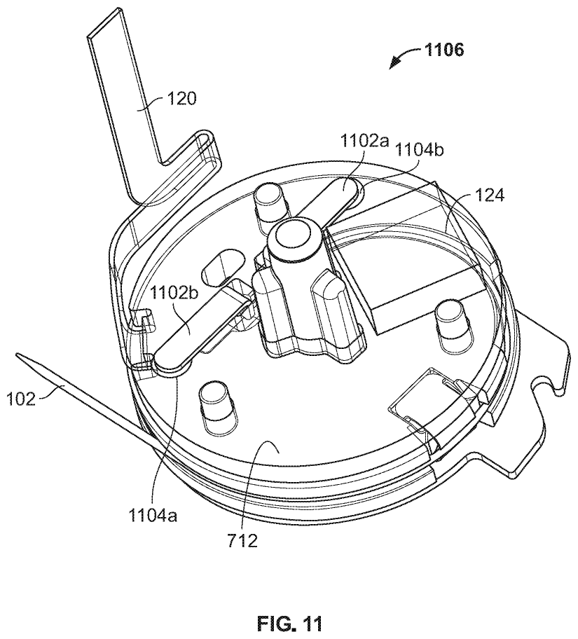

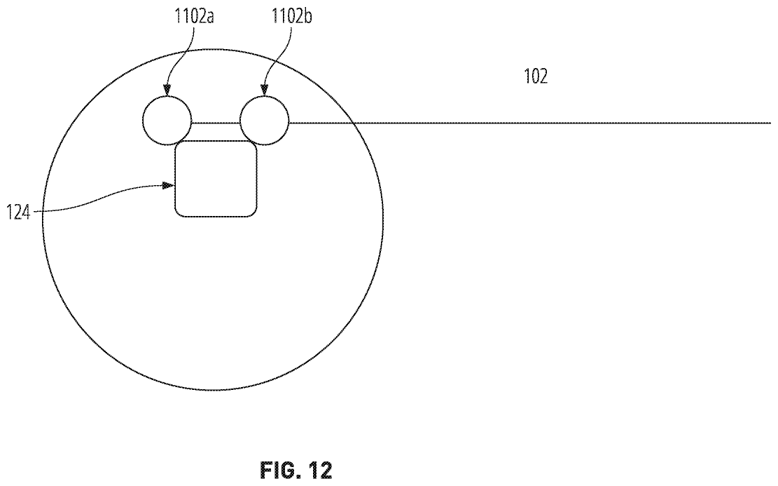

A deployable element support 712 is also mounted to the sheave 706. The deployable element support 712 includes a deployable element 102 (in this case, a sensor in the form of a laminated wire), which is wrapped circumferentially around the sheave 706 and optionally inserted into a ridge in the deployable element support 712, as shown in FIG. 11). The deployable element 102 is further constrained by the cowling 104 so that it rotates within a relatively small passage inside the deployment assembly 100. For example, the passage may be approximately 40 thousandths of an inch wide when deploying a sensor in the form of a wire that has a diameter of 4 thousandths of an inch.

As shown in the example of FIG. 1B, the structures of the cowling 104 and base 108 control the shape of this passage. The passage is configured so that the rotational motion of the deployable element 102 is converted into linear motion in the vicinity of an opening in the base 108. The opening is positioned to receive the end of the deployable element 102 as it moves linearly towards a user's skin.

The sheave 706 is mounted to a second end of the cowling 104 through a second bearing plate 710. The sheave 706 may be properly positioned with respect to the bearing plate 710 using a spacer 708.

The deployment assembly 100 may be mounted to a support 714 that may form a part of a housing or serve as a cradle or tray for the drug delivery device (e.g., unitary device housing 602) or glucose sensing device.

FIG. 8 depicts the deployable element support 712 in more detail.

The deployable element support 712 provides support for the deployable element 102 and connects the deployable element 102 to the sheave 706, properly positioning the deployable element 102 around the outer circumference of the sheave 706.

The deployable element support 712 includes a main body 802 that is circular in shape (to correspond to the shape and general size of the sheave 706). It also includes a sheave main opening 806 sized and shaped to receive a main support pillar extending from the sheave. For further support and to properly orient the deployable element support 712, the main body 802 also includes sheave subopening 804a, 804b, and 804c. These subopenings correspond to sub-supports on the sheave 706.

The deployable element support 712 may also include circuitry 808, such as embedded electronic hardware used to collect data from a sensor or control the flow of a liquid drug into a cannula. Exemplary circuitry 808 may include front-end electronics 124, as described in further detail below. In some embodiments, the deployable element 102 may include conductive traces that connect a distal portion of the deployable element 102 (e.g., the sensing portion of a sensor element) to circuitry 808 or front-end electronics 124.

In one example, the deployable element support 712, the service portion 120, and/or the deployable element 102 may be formed from a multilayer circuit board, such as a printed circuit board (PCB). One example of the makeup of the different layers of the circuit board is shown below in Table 1:

TABLE 1

| Thickness (inches) | Material | Purpose |
|---|---|---|
| 0.0005 | Polyimide | Coverlay |
| 0.0005 | Adhesive | Coverlay |
| 0.00035 | Copper | Copper Clad |
| 0.001 | Polyimide | Copper Clad |
| 0.0005 | Adhesive | Adhesive |
| 0.00035 | Copper | Copper Clad |
| 0.001 | Polyimide | Copper Clad |
| 0.00035 | Copper | Copper Clad |
| 0.005 | Adhesive | Adhesive |
| 0.001 | Polyimide | Copper Clad |
| 0.00035 | Copper | Copper Clad |
| 0.0005 | Adhesive | Coverlay |
| 0.0005 | Polyimide | Coverlay |
| 0.0005 | Adhesive | Adhesive |
| 0.001 | Stainless Steel | Stiffner |
| 0.001 | Adhesive | Coverlay |
| 0.0005 | Polyimide | Coverlay |

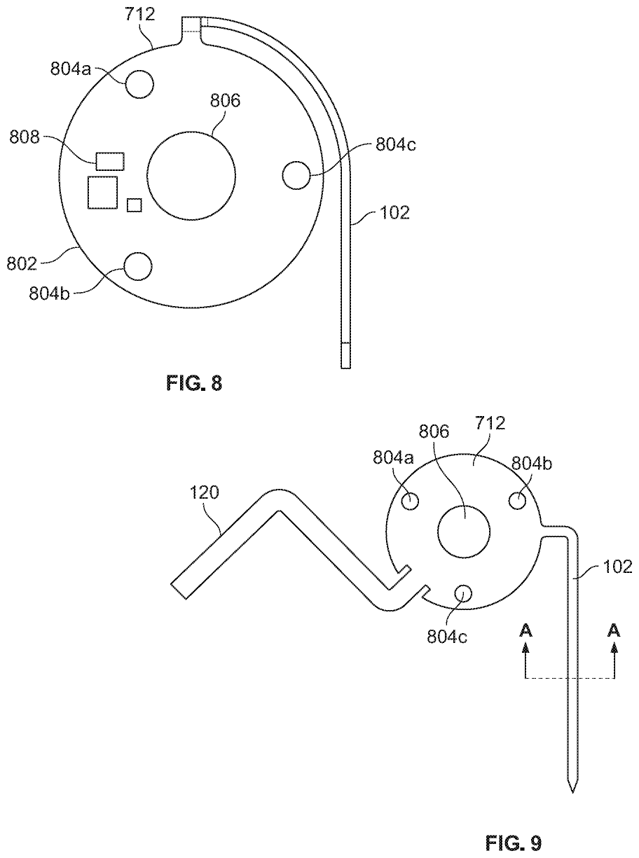

FIG. 9 depicts an alternative embodiment for the deployable element support 712. In addition to the previously-described elements, the deployable element support 712 of FIG. 9 also includes a service portion 120.

FIG. 10A and FIG. 10B depict the sheave assembly 1000 in more detail. In the sheave assembly 1000, the sheave 706 connects to the deployable element support 712 through a set of sheave supports that pass through corresponding openings in the deployable element support 712. A sheave main front support 1004 passes through the sheave main opening 806 in the deployable element support 712, while sheave front sub-support 1002a, 1002b, 1002c pass through sheave subopenings 804a, 804b, 804c. The sheave main front support 1004 then passes through the spacer 708 and is received in a corresponding opening in the bearing plate 710, which supports the sheave 706.

On the opposite side, similarly situated sub-supports pass through corresponding openings in the stop plate 112. A sheave main rear support 1008 is surrounded by the spring 704, which includes a bent end that connects through the openings in the stop plate 112 and sheave 706 to impart energy to the sheave 706. The sheave main rear support 1008 then passes into a corresponding opening in the bearing plate 702, which supports the sheave 706.

FIG. 11 is a perspective view showing electrical connections between a sensor that is contained in (or forms) the deployable element 102 and integrated front-end electronics 124, and an electrical service portion 120 for connecting the integrated front-end electronics 124 to back-end electronics in a drug delivery device main body (e.g., in a co-located device). The sensor may be integrated on the substrate of the deployable element 102 or a separate component that is electrically connected to the deployable element 102. The sensor may include a sensing element of any suitable type, such as one or more wires or microneedles.

The sensor in the deployable element 102 may be electrically connected to the deployable element support 712, and may transmit electrical sensor signals to one or more contact points 1104a, 1104b on the deployable element support 712. One or more electrically conductive contacts 1102a, 1102b may be attached to the contact points 1104a, 1104b, respectively, to transmit the signals to the integrated front-end electronics 124.

The integrated front-end electronics 124 may be, for example, a printed circuit board (PCB), integrated circuit (IC), processor, microcontroller, or other processing element. The integrated front-end electronics 124 may serve as an analog front end that amplifies a signal from the deployable element 102 and/or converts the signal into a digital form.

The integrated front-end electronics 124 may include an input configured to receive a signal from the contact points 1104a, 1104b. The input may be an analog input. The integrated front-end electronics 124 may be connected to a single deployable element 102 with one or multiple sensors, to multiple deployable elements 102 with multiple sensors, or to the deployable element 102 (with one or more sensors) and other additional types of sensing elements (independent of, or integrated with, the deployable element 102).

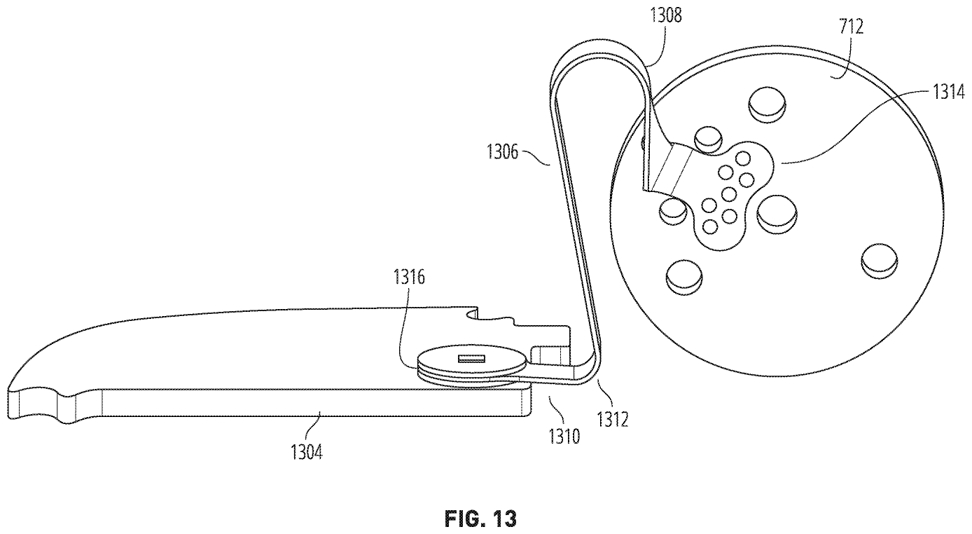

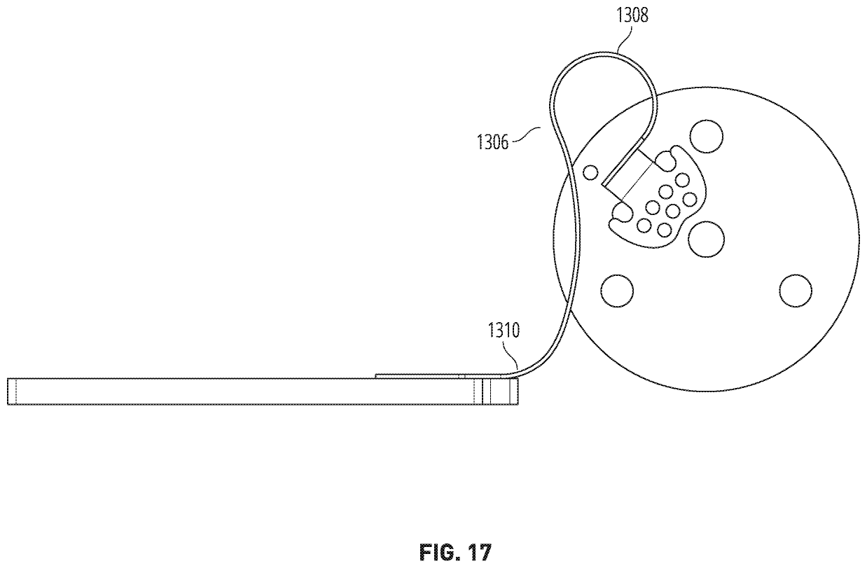

The integrated front-end electronics 124 may further include an output for transmitting a processed signal downstream towards a main processor of the body-worn device. The output may be an analog output, or may be a digital output. The output may be configured to attach to a flexible connector, as will be described in more detail in connection with FIG. 13-FIG. 21. Alternatively or in addition, the output may be a wireless transmitter, such as a radio frequency transmitter.

In some embodiments, the deployable element 102 is a sensor, such as a glucose sensor, a ketone sensor, etc. In some cases, the electrical signals from these sensors may be at a relatively low level; if sent directly to a main processor on the body-worn device, interference from the environment (e.g., in the form of radio frequency energy) may add noise to the signal to the point that the signal is too attenuated to be used by the main processor. To combat this effect, the integrated front-end electronics 124 may provide amplification of the sensor signal (e.g., through a series of stages of gains) and/or sensor bias correction, independently or in addition to other capabilities. For instance, the integrated front-end electronics 124 may provide filtering to reduce the signal-to-noise ratio (SNR) of the signal received from the deployable element 102.

Other optional features of the integrated front-end electronics 124 may include: a radio frequency (RF) transmitter/receiver or other form of wireless communication; a multiplexer and/or demultiplexer for processing multiple input sensors, as might be the case when the deployable element 102 includes a multi-analyte sensor; an accelerometer; memory; an analog-to-digital converter (ADC); a power interface for receiving electrical power from a source external to the integrated front-end electronics 124; other environmental sensors for sensing properties such as temperature, humidity, atmospheric pressure, etc; and/or a processor. The front-end electronics may also comprise a power source, such as one or more batteries, separate from other batteries on the co-located, wearable device. Alternatively, batteries elsewhere on the co-located, wearable device may power the front-end electronics 124.

If the integrated front-end electronics 124 includes memory, the memory may store suitable parameters or configuration options for the deployable element 102. For example, the configuration options may be sensor specific, including parameters specific to a particular type of sensor. In the case of a multichannel sensor signal (e.g., utilizing multiple different analytes), the memory may store configuration parameters for each type of analyte/channel. These parameters may, in some embodiments, be added to the memory of the integrated front-end electronics 124 during the manufacturing process by writing initialized parameters to the memory.

In some embodiments, the integrated front-end electronics 124 may be modular, allowing additional capabilities to be added or unused capabilities to be removed. In some cases, the size of the deployable element support 712 may dictate the number of capabilities of the integrated front-end electronics 124. Depending on the amount of available space on the integrated front-end electronics 124 and/or 712, these elements may be distributed between the integrated front-end electronics 124, the deployable element support 712, and/or a second, separate circuit board.

After initial processing, the integrated front-end electronics 124 may transmit processed data to a main processor of the body-worn device via circuitry in the service portion 120. A “main processor” is a processor configured as a central control unit of a device to process information from one or more components and to determine operation of the device based on the processed information. The service portion 120 may connect to circuitry on the body-worn device for the purpose of transferring the processed data (and/or, optionally, raw sensor signals).

This configuration allows data to be initially processed as close to the sensor as possible, which improves computation times and reduces the workload on the main processor (back-end electronics) of the body-worn device.

There are several potential problems that can arise in connection with the contact points 1104a, 1104b and the integrated front-end electronics 124. As previously noted, the electrical signals from the deployable element 102 can be subject to interference from RF energy as they are transmitted from the deployable element 102 towards the main processor of the body-worn device. Integrated front-end electronics 124 may amplify the signal from the deployable element 102, the conductive elements that carry the signals from the deployable element 102 to the integrated front-end electronics 124 can effectively function as antennas that receive RF energy and thus add noise to the signal (as can the contacts 1102a, 1102b).

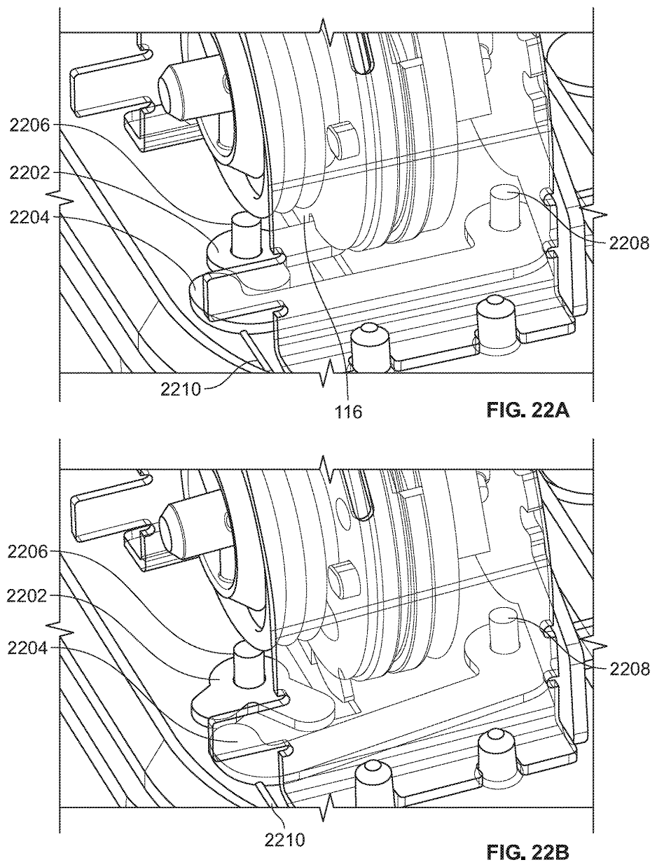

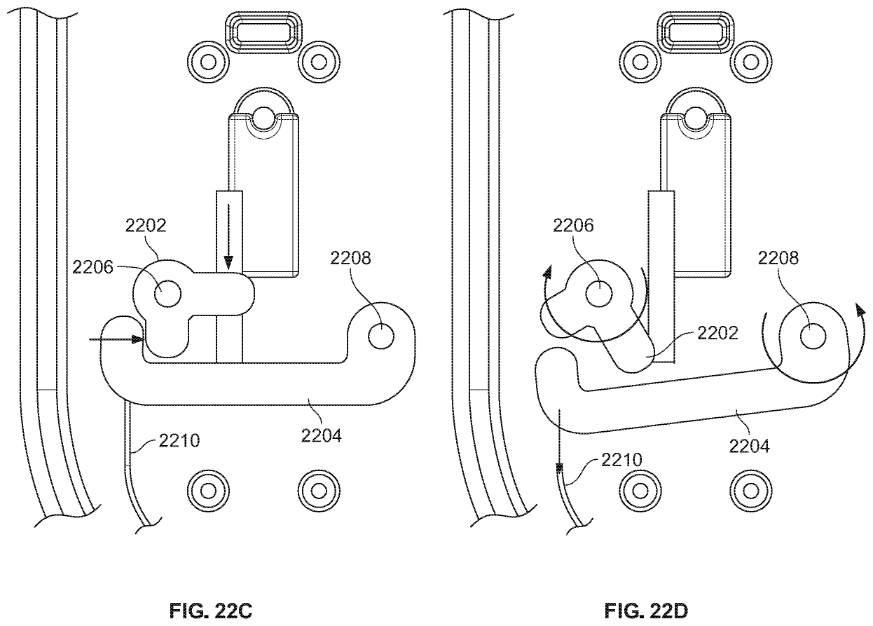

The integrated front-end electronics 124 may travel with the sensor during the deployment motion. The movement of the deployable element support 712 during sensor insertion may wake-up the integrated front-end electronics 124 from a low power or no power mode (to active mode/higher power state). A “low power state,” relative to a “higher power state” of a processor, is a state in which the processor consumes less electrical power than in the high power state, in particular by reducing or suspending execution of instructions and/or by reducing clock frequency, voltage, or activity of one or more processor components. In some embodiments, the integrated front-end electronics 124 may be woken up by closing a mechanical switch (such as the switch shown in FIG. 22A-22D), triggering an interrupt, or apply power to the circuit, among other possibilities. The front-end electronics 124 may be woken up using a wake-up circuit. A “wake-up circuit” is a circuit associated with electronics, in particular a processor and configured to operate during a reduced-power state of the electronics, to evaluate one or more input signals for a wake-up condition, and to output a wake-up signal to initiate a transition of the electronics to an operational state. In these examples, the movement of the deployable element support 712 may activate the wake-up conditions, such as by closing a mechanical switch with one end on the deployable element support 712 and another on another structure.

In the case of a switch, the switch may be the same as a switch used to wake up the main processor of the body-worn device, or may be an independent switch. FIG. 5 shows a standalone unit suitable for the use of an independent switch, while FIG. 6 shows an integrated system that could use an independent switch or the same switch as the one used by the main processor of the body-worn device. In some embodiments, a fill rod mechanism in the reservoir of the body-worn device may be used to wake up the integrated front-end electronics 124.

In some embodiments, the integrated front-end electronics 124 may also or alternatively be used to determine proper insertion of the deployable element 102 below the skin. In some cases, the deployable element 102 may not penetrate the skin or deploy into an appropriate location. For example, it is possible that the deployable element 102 buckles during deployment, preventing pentation of the skin. In another example, the deployable element 102 may strike a body structure that is harder than expected, preventing it from deploying to its full target depth. Mechanical failure of the deployment mechanism may also cause an insertion failure.

Accordingly, one or more capabilities may be provided to determine that the deployable element 102 has successfully deployed. If the above-described wakeup system employing a switch is used, then the closing of the switch may be interpreted (by the integrated front-end electronics 124 or the main processor of the body-worn device) as an indication that the rotation of the deployable element support 712 has been completed. This may serve as a rudimentary indicator of success or failure (though it may not capture all failure modes, such as the deployable element 102 buckling or the deployable element 102 not deploying to its full target depth).

In some cases, the signals received from the sensor in the deployable element 102 may be indicative of the sensor being present (or not present) in a glucose solution. For example, the sensor may include an analyte having a chemistry that is activated by the surrounding solution, and which may react differently in the presence of glucose. The integrated front-end electronics 124 and/or main processor may interpret these signals to determine whether the deployable element 102 was successfully deployed. For example, a signature of the electronic signal received from the sensor may be compared to a predetermined target signature to determine if the sensor's chemistry has been activated as expected.

If the integrated front-end electronics 124 includes an accelerometer, then information about the start position of the integrated front-end electronics 124, the end position of the integrated front-end electronics 124, and the movement profile (also referred to as deployment profile) of the integrated front-end electronics 124 in between the start and end positions over time may be collected and analyzed. A “deployment profile” is a target characteristic of a component associated with deployment, defined by at least one measurable quantity describing the movement and/or resulting state of the component. The deployment profile may in particular be defined by a deployment distance or by one or more time-dependent quantities measured during deployment, including position, velocity, acceleration, orientation, or a trajectory of the component. At the most basic level, the distance between the start and end position may be used to determine whether the deployable element support 712 rotated through the full range of expected motion. This distance may also be referred to as deployment distance. The movement profile in between the start and end positions may be compared to a target profile to detect problems such as buckling or insufficient deployment depth. A “target profile” is a predefined reference profile representing an expected characteristic of a component, wherein a deployment may be determined to be successful if an actual profile corresponds to the target profile within a predefined criterion. For instance, if the accelerometer detects insufficient acceleration, or an unexpected deceleration, this may be indicative of hitting a structure that is harder than expected in the body, which may result in the deployable element 102 being insufficiently deployed.

Alternatively or in addition, medicament delivery information may be used in conjunction with sensor data to determine whether the deployable element 102 was successfully deployed. For instance, in the case where the medicament is insulin, the system may register when insulin is delivered (e.g., when a bolus dose is requested). The signals received from the sensor after the delivery of the medicament can be compared to a predetermined profile to determine if the sensor registered an expected change (in the insulin example, a corresponding decrease in blood glucose within an expected period of time). If the sensor data does not indicate a change as was expected (represented by deviation from the predetermined profile), then the integrated front-end electronics 124 and/or main processor may determine that the sensor has not deployed as expected.

In some cases, these analyses may be employed in combination with each other. For instance, if the accelerometer indicates an abnormal deployment (actual profile not corresponding to the target profile within a predefined criterion), the system may trigger the integrated front-end electronics 124 and/or main processor to monitor the sensor data during the next bolus dose to confirm incorrect deployment.

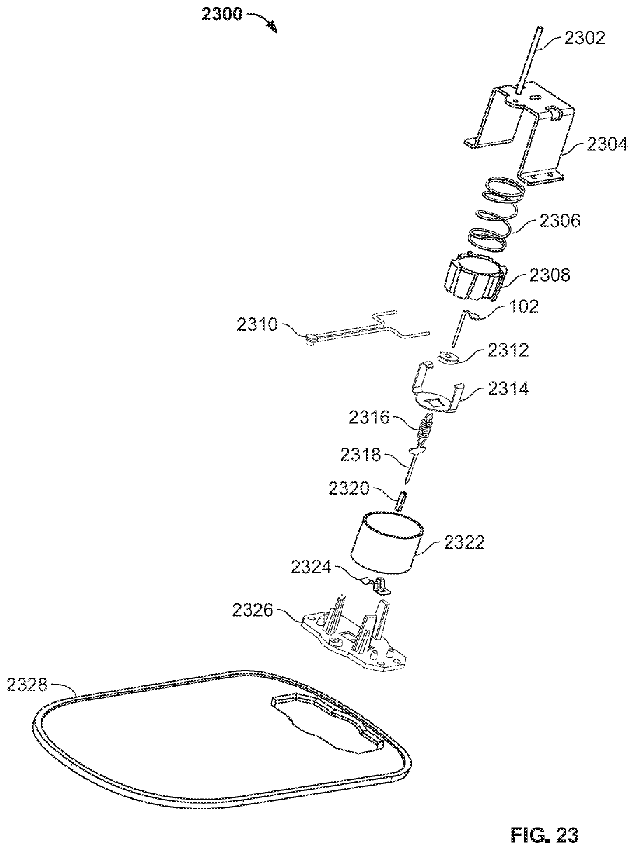

Although the integrated front-end electronics 124 in this embodiment have been described in connection with the rotary deployment mechanism discussed above, it can equally be applied to linear motion (and can therefore be used according to the same principles with the linear inserter shown in FIG. 23-FIG. 28). For instance, the deployable element 102 of FIG. 23 may be connected to a deployable element support 712 having an appropriate shape, on which integrated front-end electronics 124 are provided.

In order to move processed data from the integrated front-end electronics 124 to the main processor of the body-worn device, the output of the integrated front-end electronics 124 may be connected to another structure through a flexible connector. However, several problems can arise for this connector, which must be capable of handling the stresses that occur when the deployable element support 712 rotates and/or translates while maintaining electrical connections to the integrated front-end electronics 124 and a back-end electronics board 1304. FIG. 13-FIG. 21 depict examples of these flexible connectors and the ways in which they can connect to the front-end and back-end electronics. For the sake of clarity, FIGS. 13-19 do not depict a deployable element (e.g., a sensor element) that is configured to be deployed beneath a surface of the skin, but rather focus on, for example, how a back-end electronics board 1304 can be electrically connected to a movable deployable element support 712.

The back-end electronics may be configured to receive signals from the front-end electronics via flexible connector 1306. A “flexible connector”, as used herein, may refer to a connector configured to couple two components (here the front-end and back-end electronics) and to accommodate repeated relative movement between the components, including displacement and/or rotation, by elastic or bendable deformation of the connector while maintaining mechanical and/or electrical connection. The signals are, for example, signals from a sensor element, such as a glucose sensor, which may make up all or a portion of the deployable element 102. The signals received at the back-end electronics may have already been amplified or filtered or converted by the front-end electronics, and/or they may be amplified or filtered or converted at the back-end electronics. The back-end electronics may comprise one or more of an antenna, amplifier (fixed or programable), multiplexers, filters, memory, battery, a radio (e.g., a BLUETOOTH radio), and/or a controller or pump subsystem. The controller may use the signal to determine dosage amounts of a medicament to be delivered to a user of an automated medicament delivery device. The controller may, for example, utilize one or more algorithms that take the signals, or data indicative of a current blood glucose level of the user, and determine a medicament dosage to be delivered to the user to impact a blood glucose level of the user. The signal, or data indicative of a blood glucose level of the user, may be routed by the back-end electronics to a user device having a user interface capable of displaying the glucose level of the user.

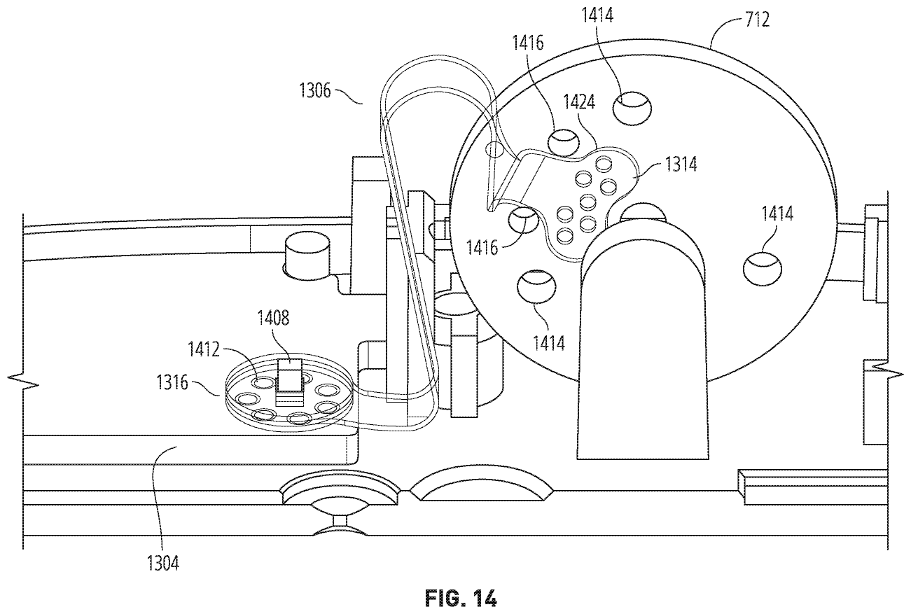

In these examples, the front-end electronics and back-end electronics are represented by rigid printed circuit boards, although other configurations are possible. The flexible connector 1306 (or numeral 120 in FIG. 1A) may be in the form of a cable, ribbon, or collection of one or more independent wires harnessed at each end. A first end 1308 of the flexible connector 1306 may connect at a proximal termination side 1314 to an output of the integrated front-end electronics 124, and a second end 1310 of the flexible connector 1306 may connect at a distal termination side 1316 to an input of a back-end electronics board 1304. The input of the back-end electronics board 1304 may be in the form of one or more contacts 1412 (see FIG. 14). The output of the integrated front-end electronics 124 may also be in the form of one or more contacts 1412. The flexible connector 1306 may be connected to the contacts 1412 by any suitable mechanism, such as solder, adhesives, etc. In one embodiment, the distal termination side 1316 and/or the proximal termination side 1314 include(s) a solder pad.

When the deployable element 102 is deployed (for example, after rotation of the deployable element support 712 (in the depicted embodiment, though linear or curvilinear deployment of the deployable element are also contemplated)), the flexible connector 1306 connecting the integrated front-end electronics 124 to the back-end electronics board 1304 undergoes strain. On the first end 1308 of the flexible connector 1306, the proximal termination side 1314 may be provided with cutouts 1424 (see FIG. 14) to accommodate heat stakes. The heat stakes can capture the flexible connector 1306 while also providing strain relief. The first end 1308 is flexible (i.e., capable of deforming elastically when subjected to stress). For instance, the flexible connector 1306 may be made of a flexible or semi-flexible material, such as polyaniline and not restricted by, for example, reinforcements that would prevent elastic deformation.

As the deployable element support 712 rotates, a portion of the flexible connector 1306 moves relative to the deployable element support 712. The heat stakes 1902 are arranged to support the flexible connector 1306 away from the solder pad connection that connects the proximal termination side 1314 to the deployable element support 712. The distance of the heat stakes 1902 away from the proximal termination side 1314 may vary depending on the specific motion that the deployable element support 712 undergoes (rotational, linear, curvilinear, etc.). It is contemplated that one or both of the integrated front-end electronics 124 and back-end electronics board 1304 may be in motion, of the same type (e.g., rotational, linear, curvilinear) or of different types. In a preferred embodiment, the front-end electronics 124 move during deployment of the deployable element while the back-end electronics 1304 remain relatively motionless during deployment.

Furthermore, it may be necessary to arrange the heat stakes so that, following the heat staking operation, the heat stakes do not cover the connection points on the deployable element support 712 (which would make it difficult to make an electrical connection between the flexible connector 1306 and the deployable element support 712 after the heat staking operation). To that end, the heat stakes 1902 may be offset to avoid the connection points.

The back-end electronics board 1304 may be in a fixed position (e.g., fixed to a housing or underbody or chassis of the body-worn device). The second end 1310 of the flexible connector 1306 may be provided with a bend 1312. Between the bend 1312 and the distal termination side 1316 of the flexible connector 1306, the flexible connector 1306 may be relatively rigid (i.e., resistant to elastic deformation). The term “rigid” as used herein refers to the property of substantially maintaining shape under expected operational forces, such that deformation is negligible for the intended function. For instance, the second end 1310 may be reinforced or formed with a different material thereby making the second end 1310 more resistant to elastic deformation than the first end 1308.

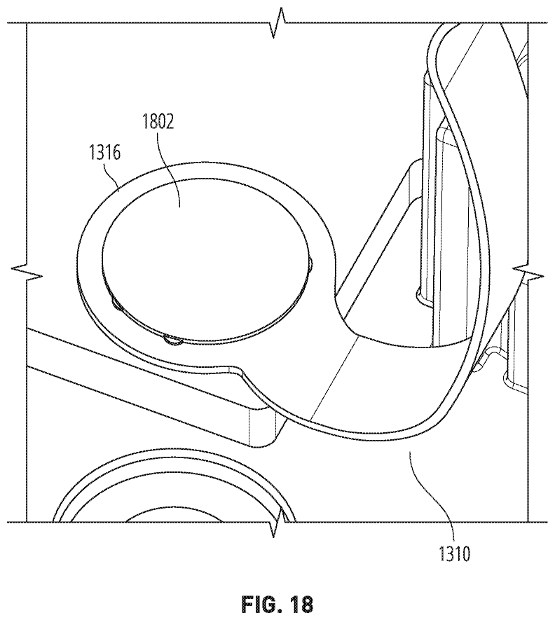

A locating peg 1408 with a square pattern may be provided to secure the distal termination side 1316 to the back-end electronics board 1304. The locating peg 1408 may be made of a suitable material for a heat staking operation so that the peg 1408 can be formed into a heat stake. When the heat staking operation is complete (see FIG. 18), the resulting distal end heat stake 1802 forces contact between the distal termination side 1316 of the flexible connector 1306 and contacts 1412 on the back-end electronics board 1304. Although the depicted peg 1408 uses a square pattern, various shapes can be used alternatively to better prevent rotating or flipping of the flexible connector 1306 during assembly.

The second end 1310 of the flexible connector 1306 may have greater robustness and can thus allow for a single heat stake (or similar securing mechanism) to capture the electrical connection and orientation between the flexible connector 1306 and back-end electronics board 1304. Thus, it may not be necessary to solder the distal termination side 1316 to the contacts 1412. This is useful in scenarios where interacting with the solder pads becomes difficult due to spacing around other components, or depth.

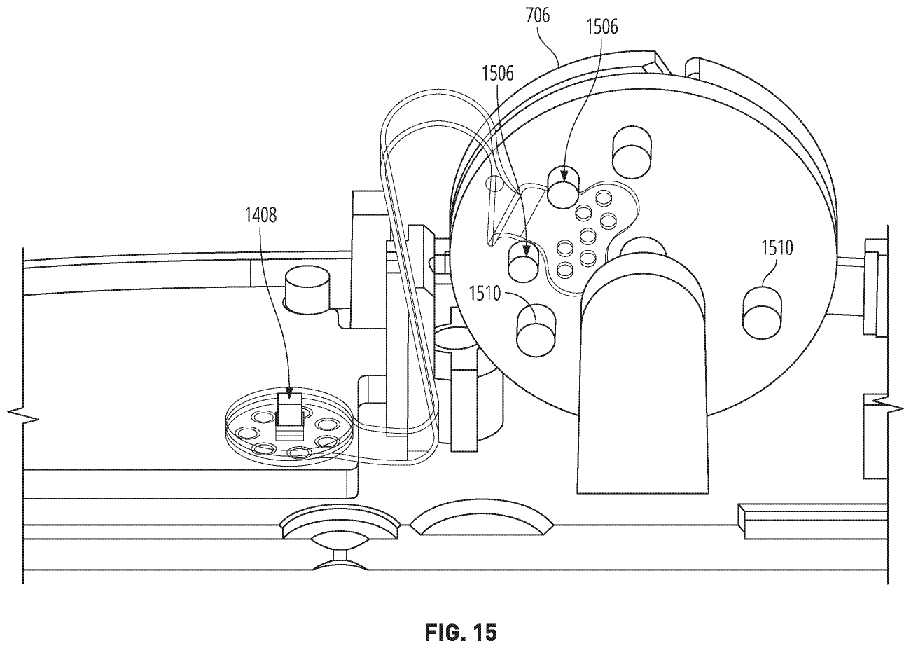

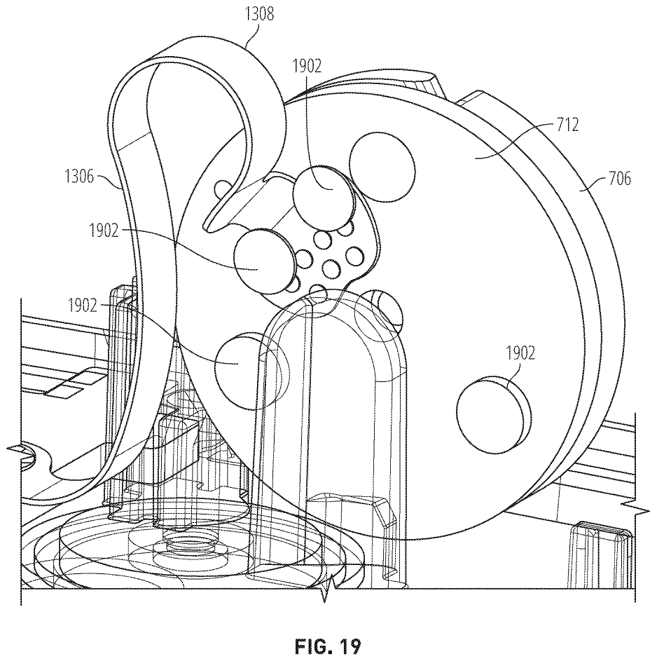

In contrast, the first end 1308 of the flexible connector 1306 attached to the deployable element support 712 utilizes two or more outer heat stakes to grab and hold the position of the flexible connector 1306, but may require individual tac welding for each solder pad on the proximal termination side 1314 to ensure a reliable connection. To situate the heat stakes, proximal termination side through holes 1416 may be provided in the deployable element support 712 proximal to cutouts 1424 in the proximal termination side 1314 of the flexible connector 1306 (when the flexible connector 1306 is positioned in its intended assembled location on the deployable element support 712). As shown in FIG. 15, distal termination side pegs 1506 may be passed through the proximal termination side through hole 1416 and then subjected to a heat staking operation to create heat stakes 1902 (see FIG. 19).

Similarly, heat stakes may also be used to fix the deployable element support 712 to the sheave 706. Through-holes 1414 may be provided through the deployable element support 712 for this purpose, through which pegs 1510 may be passed (FIG. 15) and subjected to a heat staking operation to create heat stake 1902 (FIG. 19).

In other embodiments, instead of applying multiple heat stakes 1902 on the proximal termination side 1314, a circular contact pad similar to the one used on the distal termination side 1316 may be employed on the proximal termination side 1314.

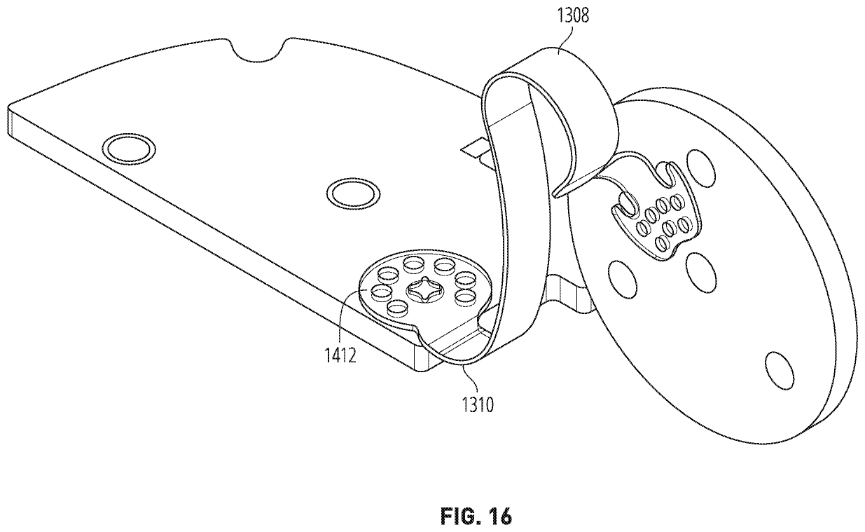

In some embodiments, the flexible connector 1306 may have two flexible ends, rather than a flexible end and a rigid end. An example of such a configuration is shown in FIG. 16 through FIG. 19. This configuration may be useful where both the integrated front-end electronics 124 and the back-end electronics board 1304 are in motion. Alternatively, providing two flexible ends may provide for cost savings and simplicity in the design of the flexible connector 1306. Such a configuration may require an added step of individually welding or joining solder pads between the flexible connector 1306 and back-end electronics board 1304 to ensure a reliable connection between the boards. A heat stake by itself may or may not be sufficient to prevent noise or bouncing during motion of the device.

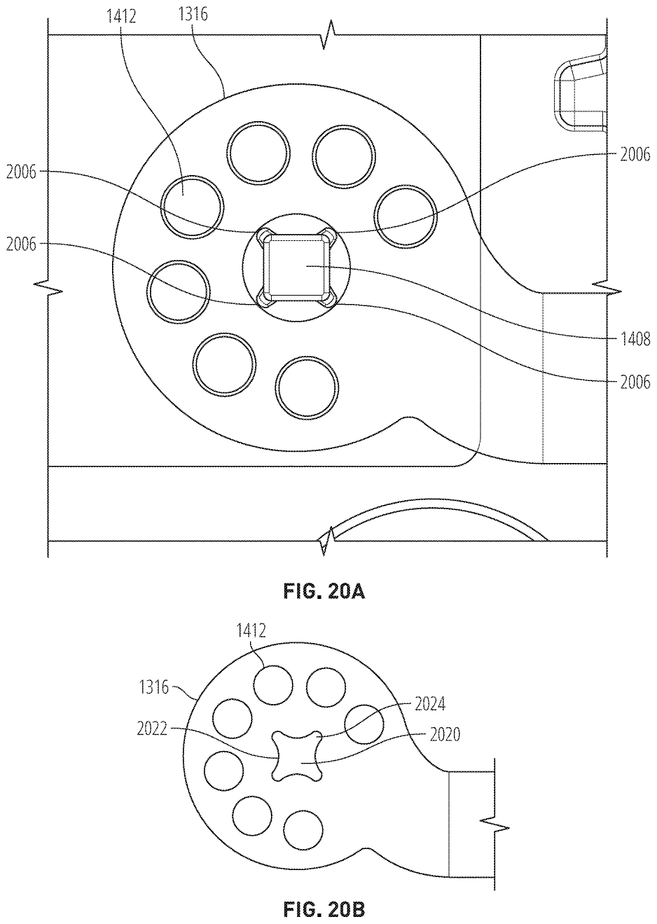

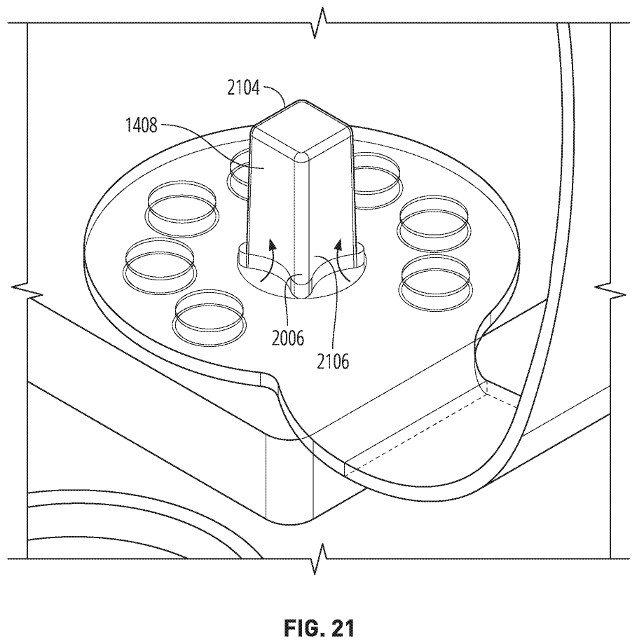

To assist with assembly of the flexible connector 1306 over an extruded boss or peg, features in the flexible connector 1306 may be provided. These features may serve to loosely position the flexible connector flat against the top of the board prior to RF welding or joining the solder pads to the board and ultimately heat-staking over the peg 1408. An example of these features is depicted in FIG. 20A-FIG. 21.

In these examples, the peg 1408 is generally square in shape, particularly at the square top 2104. The peg 1408 is positioned within an “X” shaped through-hole 2020 in the distal termination side 1410 of the flexible connector 1306. The through-hole 2020 includes curved sides 2022 and flared corners 2024. The lower portion of the peg 1408 is correspondingly shaped with concave sides 2106 and flared protrusions 2006.

As the distal termination side 1316 of the flexible connector 1306 is pressed down over the peg 1408, the interference between the flexible connector 1306 through-hole 2020 flared corners 2024 and the flared protrusions 2006 of the peg 1408 provides sufficient hold on the flexible connector 1306 prior to joining of the solder pads. Although FIG. 20A-FIG. 21 depict a particular shape to achieve this effect, numerous other examples of heat stake shapes and flexible connector 1306 shapes can also be applied to achieve the same result. The precise shape of the peg 1408 and corresponding through-hole 2020 may depend on the location, origination, and amount of stress on the peg 1408. One goal in configuring the peg 1408 is to maintain it down along the back-end electronics board 1304 instead of allowing the flexible connector 1306 and/or peg 1408 to torque. The flexible connector 1306 thus rides along the surface of the peg 1408 and then is constrained to make an electrical connection with minimal tooling.

As described above in reference to FIGS. 7A-7B, a release 110 can be pulled to release the protrusion 116 on the stop plate 112, which allows the sheave 706 to rotate due to the action of the spring. In these embodiments, the release 110 serves to hold the stop plate 112 in place until activated. FIGS. 22A-22D depict an alternative release mechanism suitable for use with exemplary embodiments, which may be useful when space is limited. In either case, a remote user device, such as a user's smartphone, can be used to trigger release, which in turn causes deployment of the deployable element 102. Thus, manual interaction with system 100 by a user may not be necessary in an embodiment where a user's remote device can be used to send a signal to communicate with electronics (e.g., the front-end electronics 124 and/or the back-end electronics 1304) within housing 602 of a wearable medical device to cause release (e.g., release of protrusion 116), which in turn causes deployment of the deployable element 102 (e.g., a glucose sensor).

In exemplary embodiments of a release mechanism, a protrusion 116 (shown in FIGS. 1B and 22A) on the stop plate 112 (FIG. 1B) is held in place by two pins mounted to the base 108. A first pin 2202 (FIG. 22A) may be L-shaped and mounted to a first pin pivot 2206. The first pin 2202 may be configured to hold the protrusion 116 in place until it swivels on the first pin pivot 2206 to move the first pin 2202 out of the way of the protrusion 116.

To that end, a second pin 2204 may be C-shaped and mounted to a second pin pivot 2208 at one end. At the other end, the second pin 2204 is attached to a release trigger 2210, such as an SMA wire.

Before the trigger 2210 is activated, the release mechanism is in the configuration shown in FIG. 22A and FIG. 22C. In this configuration, the protrusion 116 pushes on the first pin 2202 (indicated by the down arrow in FIG. 22C). The force imparted by the protrusion 116 would cause the first pin 2202 to rotate on the first pin pivot 2206, except that the first pin 2202 is prevented from rotating by the second pin 2204.

When the trigger 2210 is activated (e.g., by manually pulling on the trigger 2210, or by applying a current to the trigger 2210 when the trigger 2210 is embodied as an SMA wire, and/or by receiving a signal from a remote user device (such as a smartphone) that causes trigger 2210 to move (or contract in the case where trigger 2210 is embodied as an SMA wire)), this causes the second pin 2204 to rotate around the second pin pivot 2208 (FIG. 22D), which releases the first pin 2202. The first pin 2202 is now free to rotate round the first pin pivot 2206, thus moving the first pin 2202 away from the protrusion 116. Without the impediment of the first pin 2202, the sheave 706 is free to rotate under the action of the spring 704.

Linear Deployment Mechanism with an Optional Introducer

An alternative to the first embodiment utilizes linear, rather than rotary, movement. Examples of these embodiments are shown in FIG. 23-FIG. 26. A similar flexible cable 120, as shown in prior figures (FIG. 1A, for example) can be used in a linear deployment mechanism to connect front-end electronics on a movable deployment mechanism to back-end electronics on a non-movable portion of the wearable medical device. The discussion below will focus on the linear deployment mechanics rather than the electronic connections, which have been described above in reference to the rotary deployment mechanism.

In this embodiment, the deployable element 102 is not constrained by the cowling and base, and might therefore be more prone to buckling than the first embodiment. Accordingly, in order to stiffen the deployable element 102, an optional introducer 2318 is provided. The introducer 2318 may be placed parallel to the deployable element 102 so that the introducer 2318 supports the deployable element 102 during deployment.

However, it may be undesirable to leave the introducer 2318 embedded in the user's skin for the length of time that the deployable element 102 is deployed. This may increase the size of the entry point into the user's skin, causing discomfort (among other disadvantages). Accordingly, exemplary embodiments provide a design that allows the introducer 2318 to be automatically retracted after it has served the purpose of assisting in deploying the deployable element 102.

FIG. 23 is an exploded view showing the various mechanical parts of a linear deployment assembly 2300.

An anti-rotation pin 2302 connects a harness 2304 to the housing holding the deployment assembly 2300. The anti-rotation pin 2302 prevents the harness 2304 from rotating (e.g., due to a force imparted by a deployment spring 2306). The harness 2304 may connect, at the base of its legs, to a base 2326. The base may be sized and shaped complimentary to an opening in a base 2326 that forms the bottom of the housing in which the deployment assembly 2300 is contained.

The harness 2304 may be configured to support other elements of the deployment assembly 2300, including a deployment spring 2306, bearing cup 2308, deployable element 102, buffer plate 2312, and suspension 2314.

The deployment spring 2306 (e.g., a helical compression spring) stores and releases energy which allows the deployable element 102 to be driven linearly into the user's skin. The deployment spring 2306 pushes against a bearing cup 2308 that is sized and shaped to press against the deployable element 102, thereby transferring the energy from the deployment spring 2306 to the deployable element 102. The bearing cup 2308 is restrained by a release 2310, which prevents the deployment spring 2306 from deploying the deployable element 102 until the release 2310 is moved. The bearing cup 2308 may include protrusions that allow the introducer 2318 to be withdrawn, as described in more detail below in connection with FIG. 25.

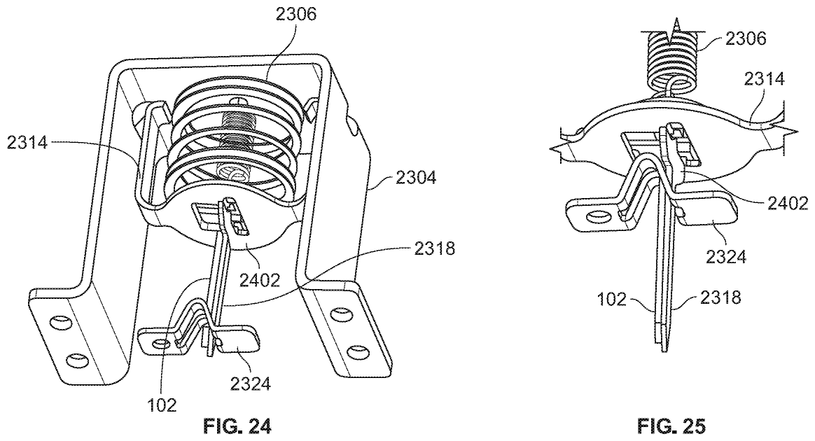

The buffer plate 2312 fits in or on a complimentary shaped opening in the suspension 2314 and supports the deployable element 102. The opening in the suspension 2314 has an opening with a first relatively narrow side and a second relatively wide side. The relatively narrow side allows the deployable element 102 and introducer 2318 to be deployed. The introducer 2318 is then pushed, due to the shape of the kick spring 2324, towards the second relatively wide side of the suspension 2314 and thereby retracts due to the action of the retraction spring 2316 (see description in connection with FIG. 24 and FIG. 25).

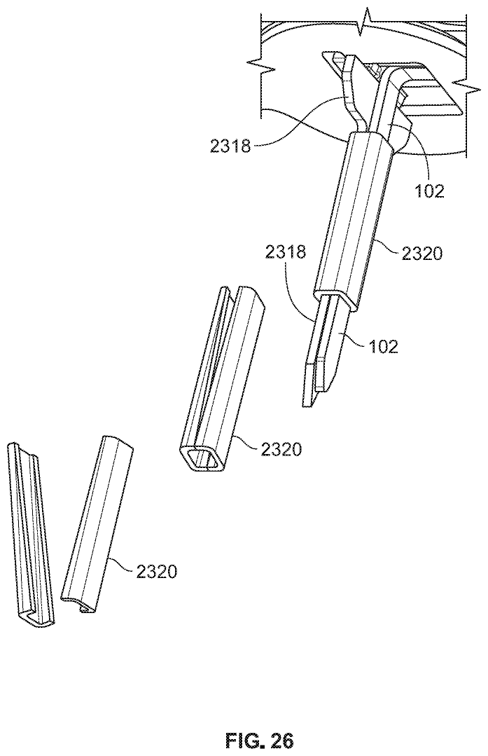

A sacrificial sleeve 2320 may temporarily hold the introducer 2318 and the deployable element 102 together, allowing them to be moved as a unit. See FIG. 26 for a more detailed description of the sacrificial sleeve 2320.

The kick spring 2324 and a support tube 2322 may be attached directly to the base 2326. The remaining elements of the deployment assembly 2300 may attach to the harness 2304, which is then affixed to the base at the bottom of the harness 2304 legs.

In operation, as shown in FIG. 24, the deployment spring 2306 is initially held in a compressed state by the release 2310 so that it stores energy. When the release 2310 is removed, the energy stored in the deployment spring 2306 is released and transmitted to the bearing cup 2308. The bearing cup 2308 in turn pushes the deployable element 102 through the opening in the suspension 2314 alongside the introducer 2318, which may be joined to the deployable element 102. This causes the combination of the deployable element 102 and introducer 2318 to translate linearly for a first predetermined distance. This action causes the retraction spring 2316 attached to the introducer 2318 to extend, thus storing energy in the retraction spring 2316.

After the first predetermined distance, both the deployable element 102 and the introducer 2318 move through the opening in the kick spring 2324. The pointed end of the introducer 2318 creates an opening in the user's skin, through which the deployable element 102 can pass.

The opening in the kick spring 2324 and the introducer 2318 are shaped in a complementary manner so that, as the introducer 2318 moves down through the opening in the kick spring 2324, the introducer is pushed towards the side of the kick spring 2324 (see FIG. 25). The introducer 2318 includes a geometric portion having an angled structure 2402 that is wider than a first portion of an opening in the suspension 2314 through which the introducer 2318 initially passes. A second portion of the opening in the suspension 2314, which is oriented towards the same side of the suspension 2314 as the direction in which the kick spring 2324 pushes the introducer 2318, has a larger opening corresponding to the size and shape of the angled structure 2402.

As the angled structure 2402 moves toward the second portion of the opening due to the interaction of the kick spring 2324 on the introducer 2318, two things happen. First, the introducer 2318 moves towards the side of the bearing cup 2308, at which location a groove is cut into the bearing cup 2308. This releases the introducer 2318 from the action of the deployment spring 2306. Second, the retraction spring 2316, which was previously restrained from pulling back on the introducer 2318 by the suspension 2314, is free to pull the introducer 2318 back above the suspension 2314. This pulls the introducer 2318 back inside the base 2326, providing it with a secure housing that prevents the pointed end of the introducer 2318 from coming back into contact with the user's skin.

As noted above, the introducer 2318 and the deployable element 102 may be joined so that they move together linearly. FIG. 26 depicts an example of a sacrificial sleeve 2320 configured to temporarily join these two elements. The sacrificial sleeve 2320 temporarily increases the structural stiffness of the elements during combined translation in one desired direction, but subsequently slices, peels, crumples, or otherwise separates from the elements upon achieving a desired amount of translation or position. This allows separate slender elements to move disconnected and differentially relative to each other in the same or opposite directions.

For example, the sacrificial sleeve 2320 may be a thin-walled element formed of polymer material. As the suspension 2314 moves down under the action of the deployment spring 2306, the sacrificial sleeve 2320 may be compressed against the kick spring 2324. At this point, it may crumple, cut, or separate along a predefined separation line. The sacrificial sleeve 2320 may remain inside the deployment assembly 2300, such as by being captured in the support tube 2322.

Similar to the embodiment described above, the deployable element 102 may be a cannula or needle for delivering a medicament such as insulin, or may be a glucose sensor or ketone sensor (e.g., an electrochemical sensor; a fiber optic sensor; a wire sensor; an optofluorescence sensor, etc.), a temperature sensor, or an activity sensor.

### Structures for Epidermal Tensioning

Because the above-described embodiments may be used with particularly thin, flexible elements, these elements may be especially susceptible to buckling or deflection. Any of these embodiments may benefit from deploying the cannulas or sensors (or the deployable element 102) into pre-tensioned tissue to reduce the chance of buckling. FIG. 27-FIG. 30 depict exemplary structures allowing the user's tissue (e.g., epidermal tissue) to be tensioned before the cannula/sensor is deployed.

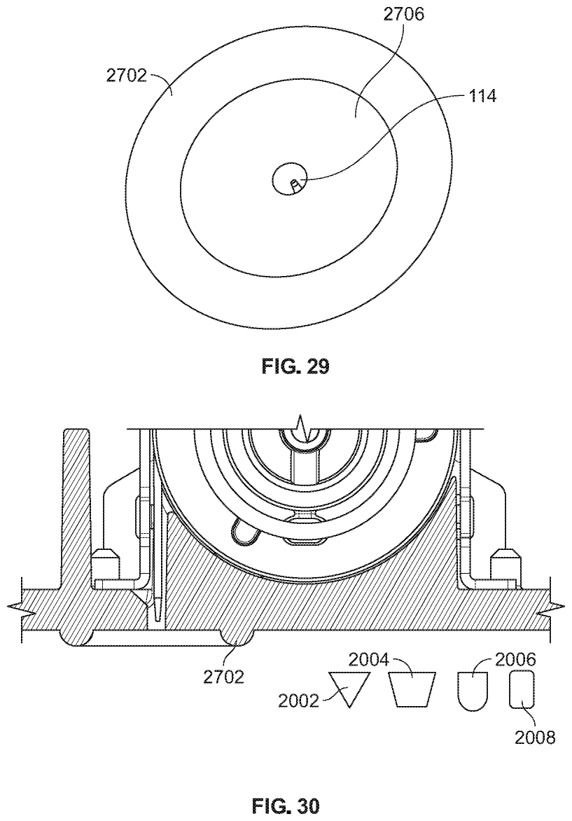

FIG. 27 and FIG. 29 depict an example of a tensioning structure that may be present on the base 108 of the deployment assembly 100. The tensioning structure includes an annular ring 2702 centered on the opening in the base of the deployment assembly 100 or deployment assembly 2300 (through which the deployable element 102 passes). The annular ring 2702 may be of any suitable size; in the depicted example, the annular ring 2702 has an outer diameter of about 0.25 inches.

The annular ring 2702 creates a gap 2706 in the center of the annular ring 2702. As the base of the deployment assembly is pressed into a user's skin 2704, the skin subtly bulges into the gap 2706, which tensions the skin 2704.

The example in FIG. 27 shows an annular ring having a semicircular cross-section. This, however, is not required, and other shapes may be suitable depending on the application. For example, FIG. 30 depicts cross sections for the annular ring having different shapes: a triangular cross-section 3002, a trapezoidal cross-section 3004, an ovoid cross-section 3006, and a rectangular cross section 3008.

FIG. 28 depicts an alternative configuration for the tensioning element. In this case, the tensioning element causes the skin 2704 to depress inwards, rather than bulging outwards as in FIG. 27. To achieve this, the bottom surface of the base 108 is projected outwards with a convex protrusion 2802 that may be centered on the opening 114. This pushes the skin 2704 away from the base 108, which causes it to pull tight against the base 108. If the deployable element 102 is being deployed at an angle, the opening 114 may be positioned such that the deployable element 102 and the tensioned skin surface where the deployable element 102 is to be inserted are nearly perpendicular to each other, or more perpendicular than they otherwise would be if the opening was at a center of the protrusion 2802.

### Exemplary Cannula and Sensor Structures

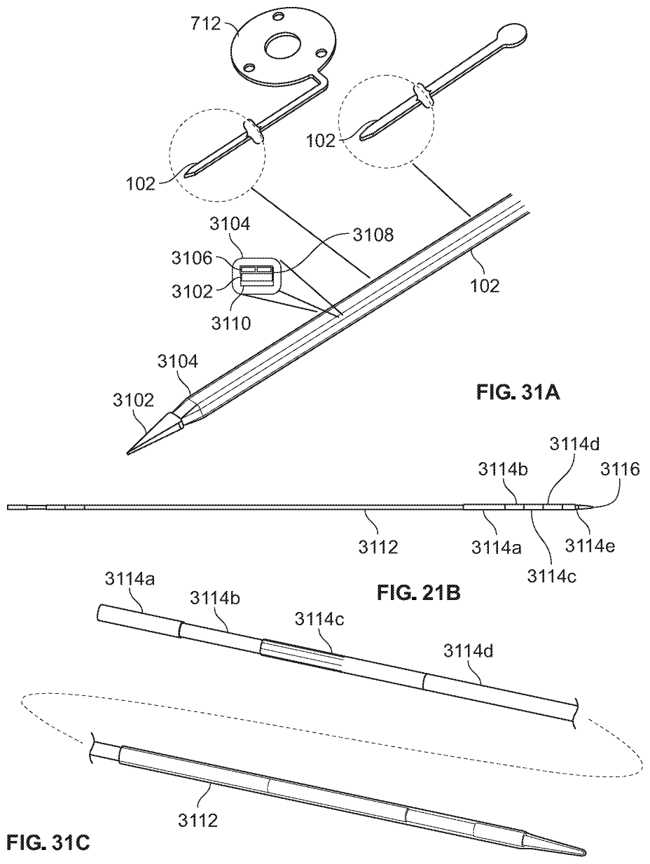

The cannula or sensor itself may also be structured or configured to support deployment, as shown in the example deployable elements 102 of FIG. 31A-FIG. 33.

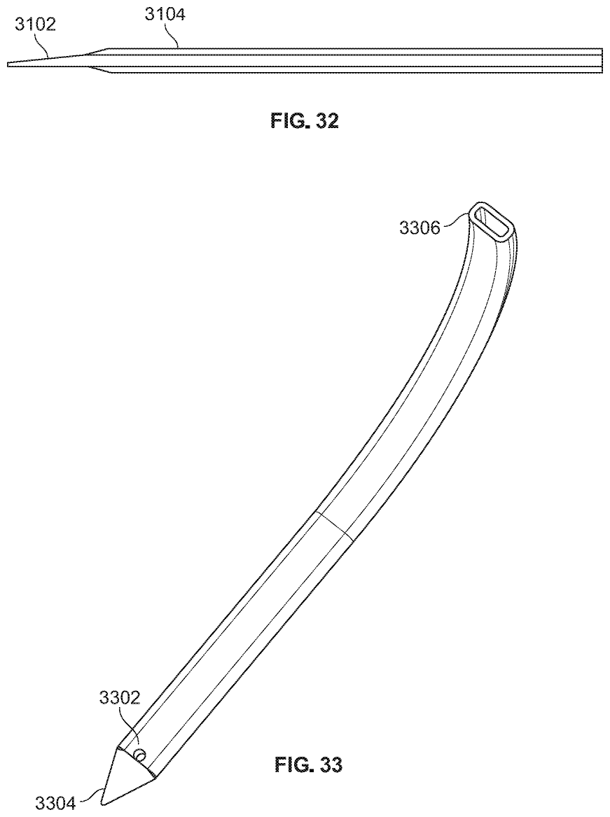

For instance, FIG. 31A-FIG. 32 depict an exemplary sensor made up of a laminated composition of polymers, metals, and other materials. These materials may be combined into varied cross-sections, and may be employed to develop desired directional stiffness (generally, although not necessarily, axial stiffness). This can improve deployment penetration of thin, flexible, and slender elements into human skin for a multi-day embedded use duration. Further it can maintain electrically conductive signal conduit/traces to embedded electronics hardware.

FIG. 31A depicts, at the top, two different deployable elements 102; the one on the left corresponds to the deployable element from the first, rotational embodiment, whereas the one on the right corresponds to the deployable element from the second, linear embodiment. The close-up at the bottom of the page represents one structure that could be suitable for use with either of these embodiments.

The deployable element 102 may be made up of a core layer 3102, an outer layer 3104, a first conductor/electrode 3106, a second conductor/electrode 3108, and/or a support 3110.

The core layer 3102 may be formed of a material that is sufficiently stiff or has a sufficiently high modulus to prevent the deployable element 102 from buckling or deflecting (e.g., stainless steel, titanium or another metal). A support 3110 may further improve the directional stiffness of the deployable element 102 for improving deployment penetration, and may be made from similar materials to the core layer 3102 and/or polymers, such as polyimides, FEP's, PTFE's, PP's, PCL's, PGA's, PGLA's, PLA's, silicone, etc. The support 3110 may also or alternatively be a biological specialty coating.

The outer layer 3104 may be a coating, such as enzymes or other bio-engineered compounds, which protects the other layers while protecting the penetration point into the user's skin. It may also be electrically insulative, thus preserving electrical signals traveling along the conductor/electrodes 3106, 3108.

The first and second conductor/electrodes 3106, 3108 may form traces that carry electrical signals from the distal end of the deployable element 102 to embedded circuitry used to process the signals. The conductor/electrodes 3106, 3108 may be made up, for example, of conductive material such as copper alloys, nickel, silver, gold, etc. The conductor/electrodes 3106, 3106 may extend along the entire length of the deployable element 102.

Once assembled, the multilayer construction may be modified at the distal tip to support penetration into the user's skin. For example, the material at the distal end could be ablated, thus removing all the material except for the core layer 3102 and allowing the core layer 3102 to be sharpened.

FIG. 31B and FIG. 31C depict additional examples of a deployable element in the form of a thin, flexible, multilayer laminated wire. In these embodiments, different layers 3114a 3114b, 3114c, 3114d, 3114e, . . . may be sequentially coated on top of a core 3116. For example, the core 3116 may be a length of wire (e.g., 5/1000s of an inch in diameter) made of tungsten or stainless steel. The layers 3114a, . . . may be insulators, conductors, enzymes, etc. coated down the length of the wire. A final layer may be a protective biocompatible coating 3112.

In some embodiments (e.g., shown in FIG. 31B), the different layers may be ablated away so that each is exposed in a sequence. Alternatively (FIG. 31C), various layers may be ablated in particular areas along the length of the wire to selectively expose them.

FIG. 33 depicts an exemplary structure suitable for a conduit. The conduit may include a strengthened tip 3304 at a distal end configured to penetrate the user's skin. The tip 3304 may be pointed or sharpened to facilitate penetration.

The conduit may be hollow to allow for a liquid drug to be dispensed through the conduit. To that end, one or more ports 3302 may be present at the distal end of the conduit in the vicinity of the tip 3304. The ports 3302 may face to the side(s) of the conduit to allow the liquid drug to be dispensed.

The conduit may, as in the depicted example, have a substantially rectangular cross section 3306. This cross section 3306 provides strength to the conduit and prevents it from collapsing, which would prevent the conduit from dispensing the liquid drug. Other suitable shapes may be used as appropriate. The proximal end of the conduit may include an interface allowing the conduit to be connected to a fluid conduit 202 that connects to a reservoir for supplying the liquid drug.

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. An apparatus comprising:
a deployable element configured to be deployed subcutaneously;
a deployable element support attached to the deployable element;
an insertion mechanism secured to the deployable element support and configured to move the deployable element support to cause the deployable element to deploy;
front-end electronics provided on the deployable element support and electrically connected to the deployable element, the front-end electronics configured to move with the deployable element support;
back-end electronics spaced apart from the deployable element support; and
a flexible connector having a first end electrically connected to the front-end electronics and movable with the deployable element support, and a second end electrically connected to the back-end electronics.

2. The apparatus of claim 1, wherein the front-end electronics comprise an accelerometer.

3. The apparatus of claim 1, wherein the deployable element comprises a sensor, and the front-end electronics comprise a memory configured to store configuration parameters for the sensor.

4. The apparatus of claim 1, wherein the sensor comprises a first sensor and a second sensor; and
the front-end electronics further comprise a multiplexer configured to multiplex a signal from the first sensor and a signal from the second sensor.

5. The apparatus of claim 1, further comprising a wake-up mechanism configured to wake up the front-end electronics from a low power state when the rotary mechanism rotates the deployable element.

6. A method comprising:
providing an apparatus according to claim 1, wherein the apparatus is initially provided in a low power state; and
triggering deployment of the deployable element by moving the deployable element, wherein moving the deployable element causes a wake-up circuit to transition the front-end electronics from the low power state to a higher power state.

7. The method of claim 6, wherein:
the apparatus is a part of a body-worn medicament delivery device comprising a main processor, and the wake-up circuit is a same wake-up circuit used to wake up the main processor from a low energy state.

8. The method of claim 6, wherein:
the apparatus is a part of a body-worn medicament delivery device comprising a main processor with a wake-up circuit configured to wake up the main processor from a low energy state, and the wake-up circuit for the front-end electronics is a different wake-up circuit than the one used to wake up the main processor from a low energy state.

9. The method of claim 6, further comprising detecting a deployment failure of the deployable element.

10. The method of claim 9, wherein detecting the deployment failure comprises detecting that the deployment failed to trigger a wake-up circuit.

11. The method of claim 9, wherein detecting the deployment failure comprises detecting that the deployable element did not move through an expected deployment distance.

12. The method of claim 9, wherein detecting the deployment failure comprises measuring a deployment profile of the deployable element throughout the deployment and comparing the deployment profile to a target deployment profile.

13. The method of claim 9, wherein detecting the deployment failure comprises examining a signal from the deployable element to determine if a sensor of the deployable element is in an activated state.

14. The method of claim 9, wherein detecting the deployment failure comprises:
detecting an expected delivery of a medicament;
examining a signal from the deployable element; and
comparing the signal from the deployable element to a predetermined profile to determine if the signal reacts to the delivery of the medicament as expected.

15. The method of claim 9, wherein detecting the deployment failure comprises:
detecting an abnormal deployment of the deployable element;
in response to detecting the abnormal deployment, triggering the front-end electronics to monitor a signal from the deployable element to determine if the signal reacts to a delivery of medicament as expected.

16. A flexible connector configured to electronically connect a first electronic structure to a second electronic structure, the first electronic structure provided on a deployable element support configured to perform at least one of rotation or translation to deliver a deployable element into a skin of a user, the flexible connector comprising:
a flexible end connected to the first electronic structure; and
a second end connected to the second electronic structure.

17. The flexible connector of claim 16, wherein the first electronic structure and the second electronic structures are rigid.

18. The flexible connector of claim 16, wherein the rotation or translation comprises curvilinear movement in three-dimensional space.

19. The flexible connector of claim 16, wherein the deployable element support is configured to rotate to deploy the deployable element, or the deployable element is configured to linearly translate to deploy the deployable element.

20. The flexible connector of claim 16, further comprising a distal termination side having a contact pad configured to connect the flexible connector to second electronic structure without the use of solder or welding, wherein the contact pad comprises a through-hole having flared corners and curved sides configured to mate with a correspondingly-shaped peg.
